SEQUENCE LISTING (177 sequences)

SEQ ID NO: 1 - protein, 10 aa, Homo sapiens
YLYDSETKNA

SEQ ID NO: 2 - protein, 9 aa, Homo sapiens
HLMDQPLSV

SEQ ID NO: 3 - protein, 9 aa, Homo sapiens
GLLKKINSV

SEQ ID NO: 4 - protein, 9 aa, Homo sapiens
FLVDGSSAL

SEQ ID NO: 5 - protein, 10 aa, Homo sapiens
FLFDGSANLV

SEQ ID NO: 6 - protein, 9 aa, Homo sapiens
FLYKIIDEL

SEQ ID NO: 7 - protein, 11 aa, Homo sapiens
FILDSAETTTL

SEQ ID NO: 8 - protein, 9 aa, Homo sapiens
SVDVSPPKV

SEQ ID NO: 9 - protein, 8 aa, Homo sapiens
VADKIHSV

SEQ ID NO: 10 - protein, 9 aa, Homo sapiens
IVDDLTINL

SEQ ID NO: 11 - protein, 9 aa, Homo sapiens
GLLEELVTV

SEQ ID NO: 12 - protein, 10 aa, Homo sapiens
TLDGAAVNQV

SEQ ID NO: 13 - protein, 10 aa, Homo sapiens
SVLEKEIYSI

SEQ ID NO: 14 - protein, 9 aa, Homo sapiens
LLDPKTIFL

SEQ ID NO: 15 - protein, 9 aa, Homo sapiens
YTFSGDVQL

SEQ ID NO: 16 - protein, 9 aa, Homo sapiens
YLMDDFSSL

SEQ ID NO: 17 - protein, 9 aa, Homo sapiens
KVWSDVTPL

SEQ ID NO: 18 - protein, 11 aa, Homo sapiens
LLWGHPRVALA

SEQ ID NO: 19 - protein, 11 aa, Homo sapiens
KIWEELSVLEV

SEQ ID NO: 20 - protein, 9 aa, Homo sapiens
LLIPFTIFM

SEQ ID NO: 21 - protein, 9 aa, Homo sapiens
FLIENLLAA

SEQ ID NO: 22 - protein, 11 aa, Homo sapiens
LLWGHPRVALA

SEQ ID NO: 23 - protein, 9 aa, Homo sapiens
FLLEREQLL

SEQ ID NO: 24 - protein, 9 aa, Homo sapiens
SLAETIFIV

SEQ ID NO: 25 - protein, 9 aa, Homo sapiens
TLLEGISRA

SEQ ID NO: 26 - protein, 9 aa, Homo sapiens
ILQDGQFLV

SEQ ID NO: 27 - protein, 10 aa, Homo sapiens
VIFEGEPMYL

SEQ ID NO: 28 - protein, 9 aa, Homo sapiens
SLFESLEYL

SEQ ID NO: 29 - protein, 9 aa, Homo sapiens
SLLNQPKAV

SEQ ID NO: 30 - protein, 9 aa, Homo sapiens
GLAEFQENV

SEQ ID NO: 31 - protein, 9 aa, Homo sapiens
KLLAVIHEL

SEQ ID NO: 32 - protein, 9 aa, Homo sapiens
TLHDQVHLL

SEQ ID NO: 33 - protein, 11 aa, Homo sapiens
TLYNPERTITV

SEQ ID NO: 34 - protein, 9 aa, Homo sapiens
KLQEKIQEL

SEQ ID NO: 35 - protein, 10 aa, Homo sapiens
SVLEKEIYSI

SEQ ID NO: 36 - protein, 11 aa, Homo sapiens
RVIDDSLVVGV

SEQ ID NO: 37 - protein, 9 aa, Homo sapiens
VLFGELPAL

SEQ ID NO: 38 - protein, 9 aa, Homo sapiens
GLVDIMVHL

SEQ ID NO: 39 - protein, 9 aa, Homo sapiens
FLNAIETAL

SEQ ID NO: 40 - protein, 9 aa, Homo sapiens
ALLQALMEL

SEQ ID NO: 41 - protein, 9 aa, Homo sapiens
ALSSSQAEV

SEQ ID NO: 42 - protein, 11 aa, Homo sapiens
SLITGQDLLSV

SEQ ID NO: 43 - protein, 9 aa, Homo sapiens
QLIEKNWLL

SEQ ID NO: 44 - protein, 9 aa, Homo sapiens
LLDPKTIFL

SEQ ID NO: 45 - protein, 9 aa, Homo sapiens
RLHDENILL

SEQ ID NO: 46 - protein, 9 aa, Homo sapiens
YTFSGDVQL

SEQ ID NO: 47 - protein, 9 aa, Homo sapiens
GLPSATTTV

SEQ ID NO: 48 - protein, 11 aa, Homo sapiens
GLLPSAESIKL

SEQ ID NO: 49 - protein, 9 aa, Homo sapiens
KTASINQNV

SEQ ID NO: 50 - protein, 9 aa, Homo sapiens
SLLQHLIGL

SEQ ID NO: 51 - protein, 9 aa, Homo sapiens
YLMDDFSSL

SEQ ID NO: 52 - protein, 9 aa, Homo sapiens
LMYPYIYHV

SEQ ID NO: 53 - protein, 9 aa, Homo sapiens
KVWSDVTPL

SEQ ID NO: 54 - protein, 11 aa, Homo sapiens
LLWGHPRVALA

SEQ ID NO: 55 - protein, 9 aa, Homo sapiens
VLDGKVAVV

SEQ ID NO: 56 - protein, 9 aa, Homo sapiens
GLLGKVTSV

SEQ ID NO: 57 - protein, 9 aa, Homo sapiens
KMISAIPTL

SEQ ID NO: 58 - protein, 11 aa, Homo sapiens
GLLETTGLLAT

SEQ ID NO: 59 - protein, 9 aa, Homo sapiens
TLNTLDINL

SEQ ID NO: 60 - protein, 9 aa, Homo sapiens
VIIKGLEEI

SEQ ID NO: 61 - protein, 9 aa, Homo sapiens
YLEDGFAYV

SEQ ID NO: 62 - protein, 11 aa, Homo sapiens
KIWEELSVLEV

SEQ ID NO: 63 - protein, 9 aa, Homo sapiens
LLIPFTIFM

SEQ ID NO: 64 - protein, 10 aa, Homo sapiens
ISLDEVAVSL

SEQ ID NO: 65 - protein, 10 aa, Homo sapiens
KISDFGLATV

SEQ ID NO: 66 - protein, 11 aa, Homo sapiens
KLIGNIHGNEV

SEQ ID NO: 67 - protein, 9 aa, Homo sapiens
ILLSVLHQL

SEQ ID NO: 68 - protein, 9 aa, Homo sapiens
LDSEALLTL

SEQ ID NO: 69 - protein, 13 aa, Homo sapiens
VLQENSSDYQSNL

SEQ ID NO: 70 - protein, 11 aa, Homo sapiens
HLLGEGAFAQV

SEQ ID NO: 71 - protein, 9 aa, Homo sapiens
SLVENIHVL

SEQ ID NO: 72 - protein, 9 aa, Homo sapiens
YTFSGDVQL

SEQ ID NO: 73 - protein, 9 aa, Homo sapiens
SLSEKSPEV

SEQ ID NO: 74 - protein, 10 aa, Homo sapiens
AMFPDTIPRV

SEQ ID NO: 75 - protein, 9 aa, Homo sapiens
FLIENLLAA

SEQ ID NO: 76 - protein, 9 aa, Homo sapiens
FTAEFLEKV

SEQ ID NO: 77 - protein, 9 aa, Homo sapiens
ALYGNVQQV

SEQ ID NO: 78 - protein, 9 aa, Homo sapiens
LFQSRIAGV

SEQ ID NO: 79 - protein, 11 aa, Homo sapiens
ILAEEPIYIRV

SEQ ID NO: 80 - protein, 9 aa, Homo sapiens
FLLEREQLL

SEQ ID NO: 81 - protein, 10 aa, Homo sapiens
LLLPLELSLA

SEQ ID NO: 82 - protein, 9 aa, Homo sapiens
SLAETIFIV

SEQ ID NO: 83 - protein, 11 aa, Homo sapiens
AILNVDEKNQV

SEQ ID NO: 84 - protein, 9 aa, Homo sapiens
RLFEEVLGV

SEQ ID NO: 85 - protein, 9 aa, Homo sapiens
YLDEVAFML

SEQ ID NO: 86 - protein, 11 aa, Homo sapiens
KLIDEDEPLFL

SEQ ID NO: 87 - protein, 9 aa, Homo sapiens
KLFEKSTGL

SEQ ID NO: 88 - protein, 11 aa, Homo sapiens
SLLEVNEASSV

SEQ ID NO: 89 - protein, 10 aa, Homo sapiens
GVYDGREHTV

SEQ ID NO: 90 - protein, 10 aa, Homo sapiens
GLYPVTLVGV

SEQ ID NO: 91 - protein, 9 aa, Homo sapiens
ALLSSVAEA

SEQ ID NO: 92 - protein, 9 aa, Homo sapiens
TLLEGISRA

SEQ ID NO: 93 - protein, 9 aa, Homo sapiens
SLIEESEEL

SEQ ID NO: 94 - protein, 9 aa, Homo sapiens
ALYVQAPTV

SEQ ID NO: 95 - protein, 10 aa, Homo sapiens
KLIYKDLVSV

SEQ ID NO: 96 - protein, 9 aa, Homo sapiens
ILQDGQFLV

SEQ ID NO: 97 - protein, 9 aa, Homo sapiens
SLLDYEVSI

SEQ ID NO: 98 - protein, 9 aa, Homo sapiens
LLGDSSFFL

SEQ ID NO: 99 - protein, 10 aa, Homo sapiens
VIFEGEPMYL

SEQ ID NO: 100 - protein, 9 aa, Homo sapiens
ALSYILPYL

SEQ ID NO: 101 - protein, 9 aa, Homo sapiens
FLFVDPELV

SEQ ID NO: 102 - protein, 11 aa, Homo sapiens
SEWGSPHAAVP

SEQ ID NO: 103 - protein, 9 aa, Homo sapiens
ALSELERVL

SEQ ID NO: 104 - protein, 9 aa, Homo sapiens
SLFESLEYL

SEQ ID NO: 105 - protein, 9 aa, Homo sapiens
KVLEYVIKV

SEQ ID NO: 106 - protein, 10 aa, Homo sapiens
VLLNEILEQV

SEQ ID NO: 107 - protein, 9 aa, Homo sapiens
SLLNQPKAV

SEQ ID NO: 108 - protein, 9 aa, Homo sapiens
KMSELQTYV

SEQ ID NO: 109 - protein, 11 aa, Homo sapiens
ALLEQTGDMSL

SEQ ID NO: 110 - protein, 11 aa, Homo sapiens
VIIKGLEEITV

SEQ ID NO: 111 - protein, 9 aa, Homo sapiens
KQFEGTVEI

SEQ ID NO: 112 - protein, 9 aa, Homo sapiens
KLQEEIPVL

SEQ ID NO: 113 - protein, 9 aa, Homo sapiens
GLAEFQENV

SEQ ID NO: 114 - protein, 9 aa, Homo sapiens
NVAEIVIHI

SEQ ID NO: 115 - protein, 9 aa, Homo sapiens
ALAGIVTNV

SEQ ID NO: 116 - protein, 12 aa, Homo sapiens
NLLIDDKGTIKL

SEQ ID NO: 117 - protein, 10 aa, Homo sapiens
VLMQDSRLYL

SEQ ID NO: 118 - protein, 9 aa, Homo sapiens
KVLEHVVRV

SEQ ID NO: 119 - protein, 9 aa, Homo sapiens
LLWGNLPEI

SEQ ID NO: 120 - protein, 9 aa, Homo sapiens
SLMEKNQSL

SEQ ID NO: 121 - protein, 9 aa, Homo sapiens
KLLAVIHEL

SEQ ID NO: 122 - protein, 10 aa, Homo sapiens
ALGDKFLLRV

SEQ ID NO: 123 - protein, 11 aa, Homo sapiens
FLMKNSDLYGA

SEQ ID NO: 124 - protein, 10 aa, Homo sapiens
KLIDHQGLYL

SEQ ID NO: 125 - protein, 12 aa, Homo sapiens
GPGIFPPPPPQP

SEQ ID NO: 126 - protein, 9 aa, Homo sapiens
ALNESLVEC

SEQ ID NO: 127 - protein, 9 aa, Homo sapiens
GLAALAVHL

SEQ ID NO: 128 - protein, 9 aa, Homo sapiens
LLLEAVWHL

SEQ ID NO: 129 - protein, 9 aa, Homo sapiens
SIIEYLPTL

SEQ ID NO: 130 - protein, 9 aa, Homo sapiens
TLHDQVHLL

SEQ ID NO: 131 - protein, 9 aa, Homo sapiens
SLLMWITQC

SEQ ID NO: 132 - protein, 11 aa, Homo sapiens
FLLDKPQDLSI

SEQ ID NO: 133 - protein, 10 aa, Homo sapiens
YLLDMPLWYL

SEQ ID NO: 134 - protein, 9 aa, Homo sapiens
GLLDCPIFL

SEQ ID NO: 135 - protein, 9 aa, Homo sapiens
VLIEYNFSI

SEQ ID NO: 136 - protein, 11 aa, Homo sapiens
TLYNPERTITV

SEQ ID NO: 137 - protein, 9 aa, Homo sapiens
AVPPPPSSV

SEQ ID NO: 138 - protein, 9 aa, Homo sapiens
KLQEELNKV

SEQ ID NO: 139 - protein, 11 aa, Homo sapiens
KLMDPGSLPPL

SEQ ID NO: 140 - protein, 9 aa, Homo sapiens
ALIVSLPYL

SEQ ID NO: 141 - protein, 9 aa, Homo sapiens
FLLDGSANV

SEQ ID NO: 142 - protein, 10 aa, Homo sapiens
ALDPSGNQLI

SEQ ID NO: 143 - protein, 9 aa, Homo sapiens
ILIKHLVKV

SEQ ID NO: 144 - protein, 9 aa, Homo sapiens
VLLDTILQL

SEQ ID NO: 145 - protein, 9 aa, Homo sapiens
HLIAEIHTA

SEQ ID NO: 146 - protein, 9 aa, Homo sapiens
SMNGGVFAV

SEQ ID NO: 147 - protein, 9 aa, Homo sapiens
MLAEKLLQA

SEQ ID NO: 148 - protein, 9 aa, Homo sapiens
YMLDIFHEV

SEQ ID NO: 149 - protein, 11 aa, Homo sapiens
ALWLPTDSATV

SEQ ID NO: 150 - protein, 9 aa, Homo sapiens
GLASRILDA

SEQ ID NO: 151 - protein, 9 aa, Homo sapiens
ALSVLRLAL

SEQ ID NO: 152 - protein, 9 aa, Homo sapiens
SYVKVLHHL

SEQ ID NO: 153 - protein, 9 aa, Homo sapiens
VYLPKIPSW

SEQ ID NO: 154 - protein, 9 aa, Homo sapiens
NYEDHFPLL

SEQ ID NO: 155 - protein, 9 aa, Homo sapiens
VYIAELEKI

SEQ ID NO: 156 - protein, 12 aa, Homo sapiens
VHFEDTGKTLLF

SEQ ID NO: 157 - protein, 9 aa, Homo sapiens
VLSPFILTL

SEQ ID NO: 158 - protein, 9 aa, Homo sapiens
HLLEGSVGV

SEQ ID NO: 159 - protein, 9 aa, Homo sapiens
ALREEEEGV

SEQ ID NO: 160 - protein, 10 aa, Homo sapiens
KEADPTGHSY

SEQ ID NO: 161 - protein, 9 aa, Homo sapiens
TLDEKVAEL

SEQ ID NO: 162 - protein, 9 aa, Homo sapiens
KIQEILTQV

SEQ ID NO: 163 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 164 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 165 - protein, 226 aa, synthetic construct
  REGION 1..226: note = m1CD8alpha
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPASVVDFLPTTAQPTKKSTLKKRVCRLPRPETQKGPLCSPIYIWAPLAGTCGVLLLSLVITLYCNHRNRRRVCKCPRPVVKSGDKPSLSARYV

SEQ ID NO: 166 - protein, 226 aa, synthetic construct
  REGION 1..226: note = m2CD8alpha
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPASVVDFLPTTAQPTKKSTLKKRVCRLPRPETQKGPLCSPIYIWAPLAGTCGVLLLSLVITLYCNHRNRRRVCKCPRPVVKSGDKPSLSARYV

SEQ ID NO: 167 - protein, 243 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQPQGEGISGTFVPQCLHGYYSNTTTSQKLLNPWILKT

SEQ ID NO: 168 - protein, 198 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGLKGKVYQEPLSPNACMDTTAILQPHRSCLTHGS

SEQ ID NO: 169 - protein, 189 aa, Homo sapiens
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYK

SEQ ID NO: 170 - protein, 200 aa, Homo sapiens
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQLRLHPLEKCSRMDY

SEQ ID NO: 171 - protein, 225 aa, Homo sapiens
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQKFNIVCLKISGFTTCCCFQILQISREYGFGVLLQKDIGQ

SEQ ID NO: 172 - protein, 225 aa, Homo sapiens; identical to SEQ ID NO: 171

SEQ ID NO: 173 - protein, 222 aa, Homo sapiens
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQPQGEGISGTFVPQCLHGYYSNTTTSQKLLNPWILKT

SEQ ID NO: 174 - DNA, 607 bp, synthetic construct
  misc_feature 1..607: note = WPRE
  source 1..607: mol_type = other DNA
cagtctgacgtacgcgtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcc

SEQ ID NO: 175 - DNA, 607 bp, synthetic construct
  misc_feature 1..607: note = WPREmut1
  source 1..607: mol_type = other DNA
cagtctgacgtacgcgtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcc

SEQ ID NO: 176 - DNA, 581 bp, synthetic construct
  misc_feature 1..581: note = WPREmut2
  source 1..581: mol_type = other DNA
gagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 177 - protein, 559 aa, synthetic construct
  REGION 1..559: note = RD114TR
MKLPTGMVILCSLIIVRAGFDDPRKAIALVQKQHGKPCECSGGQVSEAPPNSIQQVTCPGKTAYLMTNQKWKCRVTPKISPSGGELQNCPCNTFQDSMHSSCYTEYRQCRRINKTYYTATLLKIRSGSLNEVQILQNPNQLLQSPCRGSINQPVCWSATAPIHISDGGGPLDTKRVWTVQKRLEQIHKAMTPELQYHPLALPKVRDDLSLDARTFDILNTTFRLLQMSNFSLAQDCWLCLKLGTPTPLAIPTPSLTYSLADSLANASCQIIPPLLVQPMQFSNSSCLSSPFINDTEQIDLGAVTFTNCTSVANVSSPLCALNGSVFLCGNNMAYTYLPQNWTRLCVQASLLPDIDINPGDEPVPIPAIDHYIHRPKRAVQFIPLLAGLGITAAFTTGATGLGVSVTQYTKLSHQLISDVQVLSGTIQDLQDQVDSLAEVVLQNRRGLDLLTAEQGGICLALQEKCCFYANKSGIVRNKIRTLQEELQKRRESLASNPLWTGLQGFLPYLLPLLGPLLTLLLILTIGPCVFNRLVQFVKDRISVVQALVLTQQYHQLKPL

(19) United States
(12) Patent Application Publication
(10) Pub. No.: US 2023/0089392 A1
(43) Pub. Date: Mar. 23, 2023

(54) MONOCYTE DEPLETION OF T CELLS POPULATIONS FOR T-CELL THERAPY

(71) Applicant: Immatics US, Inc., Houston, TX (US)

(72) Inventors: Justin GUNESCH, Houston, TX (US); Pooja MEHTA, Houston, TX (US); Mamta KALRA, Houston, TX (US)

(21) Appl. No.: 17/933,418

(22) Filed: Sep. 19, 2022

Related U.S. Application Data
(60) Provisional application No. 63/395,663, filed on Aug. 5, 2022.
(60) Provisional application No. 63/246,179, filed on Sep. 20, 2021.

(51) Int. Cl. C12N 5/0783; C07K 14/725; C07K 14/705; C07K 14/74

(52) U.S. Cl. CPC C12N 5/0636; C07K 14/7051; C07K 14/70517; C07K 14/70539

(57) ABSTRACT

A method for producing an engineered T cell population includes obtaining a cell population containing a monocyte and a T cell, resting the obtained cell population on a surface, adhering the monocyte to the surface, retaining a non-adherent cell population, activating the non-adherent cell population, introducing a nucleic acid into the activated non-adherent cell population to obtain a transformed T cell, and expanding the transformed T cell to obtain the engineered T cell population.

### CROSS-REFERENCE TO RELATED APPLICATIONS

This application claims priority to U.S. No. 63/246,179, filed 20 Sep. 2021, and U.S. No. 63/395,663, filed 5 Aug. 2022, which are incorporated herein by reference in their entireties.

### REFERENCE TO SEQUENCE LISTING SUBMITTED AS A COMPLIANT ASCII TEXT FILE (.xml)

Pursuant to the EFS-Web legal framework and 37 C.F.R. § 1.821-825 (see M.P.E.P. § 2442.03(a)), a Sequence Listing in the form of an ASCII-compliant text file (entitled “3000011-025002_Sequence_Listing_ST26.xml” created on Sep. 19, 2022, and 159,708 bytes in size) is submitted concurrently with the instant application, and the entire contents of the Sequence Listing are incorporated herein by reference.

### FIELD OF THE INVENTION

The present disclosure generally relates to methods of manufacturing T cells for adoptive immunotherapy comprising a step of depleting adherent cells, including but not limited to monocytes. The disclosure further provides for methods of genetically transducing the T cells isolated by the methods described herein, methods of using the T cells, and T cell populations thereof.

### BACKGROUND OF THE INVENTION

In adoptive cell therapy, lymphocytes isolated from a patient are genetically modified ex vivo to express recombinant proteins that enable the cells to perform new therapeutic functions after subsequently transfer back into the patient. For example, T cells may be isolated from the lymphocytes and genetically modified to express a recombinant chimeric antigen receptor (“CAR T cells”) and/or a T-cell receptor (“TCR therapy”). In CAR T cell therapy, the cells recognize antigens expressed on the surface of cells, whereas TCR therapy cells recognize tumor-specific proteins inside the cells, presented on the surface in an WIC complex. TCR cells are generally engineered to recognize a tumor-specific antigen/WIC combination.

Before the modified T cells are transferred back into the patient, the modified T cells are expanded ex vivo to create a sufficient number of cells to achieve a therapeutic effect. When lymphocytes isolated and returned to the same patient it is generally referred to as “autologous cell therapy”. When the lymphocytes are isolated from a compatible donor and infused into a new, different patient, the process is generally referred to as “allogenic cell therapy.”

There is a need in the art for a rapid, streamlined, and safe method to isolate lymphocytes, genetically modify and expand the genetically modified lymphocytes ex vivo. Such methods may expand the deployment of methods of adoptive cell therapy, such as chimeric antigen receptor technologies (CAR-T) and T cell receptor technologies (TCR-T), which may hold promise for many patients who currently are in need of an effective cancer treatment.

### SUMMARY OF VARIOUS EMBODIMENTS OF THE INVENTION

The present disclosure relates to methods of methods for producing a cytotoxic T lymphocyte (CTL) comprising (a) obtaining a population of peripheral blood mononuclear cells (PMBC); (b) depleting the adherent cells, optionally monocytes; (c) isolating T cells from peripheral blood mononuclear cells (PBMC), (b) activating the isolated T cells with an anti-CD3 antibody and an anti-CD28 antibody, (c) introducing a nucleic acid into the activated T cells, (d) expanding the transformed T cells, and (e) harvesting the transformed CD8+ T cells, wherein step (a) through the step (e) are performed within 6 days. In another aspect, the method takes no longer than 6 days to complete. The method may take 1, 2, 3, 4, 5, 6, or 7 days or more to complete. The method may further comprise cryopreserving the harvested T-cells. In an embodiment, the T-cells may be CD8+ T-cells, CD4+, and/or γδ T cells.

In an embodiment, a method for depleting monocytes from a population of peripheral blood mononuclear cells (PMBC) may comprise resting the PMBC in a vessel for a time sufficient for portion of the monocytes to adhere to the vessel and removing the non-adherent cells.

In an embodiment, a method for depleting adherent cells from a population of peripheral blood mononuclear cells (PMBC) may comprise resting the PMBC in a vessel for a time sufficient for portion of the cells to adhere to the vessel and removing the non-adherent cells. The adherent cells may comprise monocytes.

In an embodiment, the adherent cells may be depleted from the PMBC population. The adherent cells may be depleted by a method comprises resting the PMBC in a vessel for a time sufficient for portion of the cells to adhere to the vessel and removing the non-adherent cells.

In an embodiment, the time sufficient for portion of the cells to adhere to the vessel may be between about 1 and 10 hours. The time may be about 1, 2, 3, 4, 5, 6, 7, 8, 9, or 10 hours. The time may be between about 1-6 hours, about 2-4 hours, about 1-4 hours, about 3-6 hours, about 4-10 hours, or about 2-3 hours.

In an embodiment, the cell culture vessel may be plastic or glass. The plastic may be polysterene or polycarbonate. The vessel may be treated with a coating.

In an embodiment, the PMBC may be seeded in the vessel at a density of cells between about 0.1 and 2.0 million cells/cm². The density of the cells may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 million cells/cm². The density of the cells may be about 0.3, 0.5, 0.8, or 1.2 million cells/cm². The density of the cells may be from about 0.1 to 2.0 million cells/cm², from about 0.3 to 1.0 million cells/cm², from about 0.5 to 0.8 million cells/cm², from about 0.5 to 1.0 million cells/cm², from about 0.3 to 1.5 million cells/cm², from about 0.8 to 2.0 million cells/cm², or from about 1.0 to 2.0 million cells/cm².

In an embodiment, the cell culture vessel may be a flask, dish, bag, cellstack, or an assemblage thereof. The cell culture vessel may comprise a plurality of flasks, dishes, bags, cellstacks, or an assemblage thereof.

In an embodiment, cellstacks may include at least 1, at least 2, at least 3, at least 4, at least 5, at least 6, at least 7, at least 8, at least 9, at least 10, at least 15, at least 20, at least 25, at least 30, at least 35, at least 40, at least 45, or at least 50 stacks.

In another embodiment, cellstacks may have at least 400, at least 500, at least 600, at least 700, at least 800, at least 900, at least 1,000, at least 2,000, at least 3,000, at least 4,000, at least 5,000, at least 6,000, at least 7,000, at least 8,000, at least 9,000, at least 10,000, at least 20,000, at least 30,000, at least 40,000, or at least 50,000 cm² total cell growth area.

In another embodiment, 1-stack may have about 636 cm² cell growth area, 2-stack may have about 1,272 cm² cell growth area, 5-stack may have about 3,180 cm² cell growth area, 10-stack may have about 6,360 cm² cell growth area, and 40-stack may have about 25,440 cm² cell growth area.

In another embodiment, cellstacks may be obtained from Corning®, GBO®, VWR®, or Nunc™.

In an embodiment, the adherent cells may comprise monocytes. The adherent cells depleted by the method may comprise myeloid derived suppressor cells (MDSCs), e.g., MDSC1, MDSC2, and MDSC7 subsets.

In an embodiment, the peripheral blood mononuclear cells (PBMC) may be obtained from a healthy donor. The peripheral blood mononuclear cells (PBMC) may be obtained from a patient.

In an embodiment, the number of the isolated T cells may be from about 1×10⁸ to about 3×10⁹, from about 2×10⁸ to about 3×10⁹, from about 3×10⁸ to about 3×10⁹, from about 4×10⁸ to about 3×10⁹, from about 5×10⁸ to about 3×10⁹, from about 6×10⁸ to about 3×10⁹, from about 7×10⁸ to about 3×10⁹, from about 8×10⁸ to about 3×10⁹, from about 9×10⁸ to about 3×10⁹, from about 1×10⁹ to about 3×10⁹, from about 1×10⁹ to about 2.5×10⁹, from about 1×10⁹ to about 2×10⁹, or from about 1×10⁹ to about 1.5×10⁹. The number of the isolated T cells may be about 1×10⁸ cells, about 2×10⁸ cells, about 3×10⁸ cells, about 4×10⁸ cells, about 5×10⁸ cells, about 6×10⁸ cells, about 7×10⁸ cells, about 8×10⁸ cells, about 9×10⁸ cells, about 1×10⁹ cells, about 2×10⁹ cells, about 3×10⁹ cells, about 4×10⁹ cells, about 5×10⁹ cells, about 6×10⁹ cells, about 7×10⁹ cells, about 8×10⁹ cells, about 9×10⁹ cells, or about 1×10¹⁰ cells. In an embodiment, the T-cells may be CD8+ T-cells, CD4+, and/or γδ T cells.

In an embodiment, the purity of the isolated T cells in a preparation may be from about 60% to about 100%, from about 65% to about 100%, from about 70% to about 100%, from about 75% to about 100%, from about 80% to about 100%, from about 85% to about 100%, from about 90% to about 100%, from about 95% to about 100%, from about 96% to about 100%, from about 97% to about 100%, from about 98% to about 100%, or from about 99% to about 100%. The purity of the isolated T cells in a preparation may be about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. In an embodiment, the T-cells may be CD8+ T-cells, CD4+, and/or γδ T cells.

In an embodiment, the anti-CD3 antibody may be in a concentration of from about 0.1 μg/ml to about 10.0 μg/ml, about 0.1 μg/ml to about 8.0 μg/ml, about 0.1 μg/ml to about 6.0 μg/ml, about 0.1 μg/ml to about 4.0 μg/ml, about 0.1 μg/ml to about 2.0 μg/ml, about 0.1 μg/ml to about 1.0 μg/ml, about 0.1 μg/ml to about 0.8 μg/ml, about 0.1 μg/ml to about 0.6 μg/ml, about 0.1 μg/ml to about 0.5 μg/ml, about 0.1 μg/ml to about 0.25 μg/ml, about 0.2 μg/ml to about 0.5 μg/ml, about 0.2 μg/ml to about 0.3 μg/ml, about 0.3 μg/ml to about 0.5 μg/ml, about 0.3 μg/ml to about 0.4 μg/ml, or about 0.4 μg/ml to about 0.5 μg/ml. The anti-CD3 antibody may be in a concentration of about 0.1, 0.2, 0.3, 0.4, 0.5, 0.6, 0.7, 0.8, 0.9, 1, 2, 3, 4, 5, 6, 7, 8, 9, or 10 μg/ml.

In an embodiment, the anti-CD28 antibody may be in a concentration of from about 0.1 μg/ml to about 10.0 μg/ml, about 0.1 μg/ml to about 8.0 μg/ml, about 0.1 μg/ml to about 6.0 μg/ml, about 0.1 μg/ml to about 4.0 μg/ml, about 0.1 μg/ml to about 2.0 μg/ml, about 0.1 μg/ml to about 1.0 μg/ml, about 0.1 μg/ml to about 0.8 μg/ml, about 0.1 μg/ml to about 0.6 μg/ml, about 0.1 μg/ml to about 0.5 μg/ml, about 0.1 μg/ml to about 0.25 μg/ml, about 0.2 μg/ml to about 0.5 μg/ml, about 0.2 μg/ml to about 0.3 μg/ml, about 0.3 μg/ml to about 0.5 μg/ml, about 0.3 μg/ml to about 0.4 μg/ml, or about 0.4 μg/ml to about 0.5 μg/ml. The anti-CD28 antibody may be in a concentration of about 0.1, 0.2, 0.3, 0.4, 0.5, 0.6, 0.7, 0.8, 0.9, 1, 2, 3, 4, 5, 6, 7, 8, 9, or 10 μg/ml.

In an embodiment, both the anti-CD3 antibody and the anti-CD28 antibody may each be in a concentration of from about 0.1 μg/ml to about 10.0 μg/ml, about 0.1 μg/ml to about 8.0 μg/ml, about 0.1 μg/ml to about 6.0 μg/ml, about 0.1 μg/ml to about 4.0 μg/ml, about 0.1 μg/ml to about 2.0 μg/ml, about 0.1 μg/ml to about 1.0 μg/ml, about 0.1 μg/ml to about 0.8 μg/ml, about 0.1 μg/ml to about 0.6 μg/ml, about 0.1 μg/ml to about 0.5 μg/ml, about 0.1 μg/ml to about 0.25 μg/ml, about 0.2 μg/ml to about 0.5 μg/ml, about 0.2 μg/ml to about 0.3 μg/ml, about 0.3 μg/ml to about 0.5 μg/ml, about 0.3 μg/ml to about 0.4 μg/ml, or about 0.4 μg/ml to about 0.5 μg/ml. The both the anti-CD3 antibody and the anti-CD28 antibody may be in a concentration of about 0.1, 0.2, 0.3, 0.4, 0.5, 0.6, 0.7, 0.8, 0.9, 1, 2, 3, 4, 5, 6, 7, 8, 9, or 10 μg/ml. In one embodiment, the concentration of the combination of the anti-CD3 antibody and the anti-CD28 antibody may be in a concentration of about 0.1, 0.2, 0.3, 0.4, 0.5, 0.6, 0.7, 0.8, 0.9, 1, 2, 3, 4, 5, 6, 7, 8, 9, or 10 μg/ml.

In an embodiment, the activation of the T cells may be completed within a period of from about 1 hour to about 120 hours, about 1 hour to about 108 hours, about 1 hour to about 96 hours, about 1 hour to about 84 hours, about 1 hour to about 72 hours, about 1 hour to about 60 hours, about 1 hour to about 48 hours, about 1 hour to about 36 hours, about 1 hour to about 24 hours, about 2 hours to about 24 hours, about 4 hours to about 24 hours, about 6 hours to about 24 hours, about 8 hours to about 24 hours, about 10 hours to about 24 hours, about 12 hours to about 24 hours, about 12 hours to about 72 hours, about 24 hours to about 72 hours, about 6 hours to about 48 hours, about 24 hours to about 48 hours, about 6 hours to about 72 hours, or about 1 hours to about 12 hours. The activation of the T cells may be completed in about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 hours. The activation of the T cells may be carried for about 1-10 hours, 11-30 hours, 31-50 hours, 51-100 hours, or 101-120 hours. In an embodiment, the T-cells may be CD8+ T-cells, CD4+, and/or γδ T cells.

In an embodiment, the anti-CD3 antibody, the anti-CD28 antibody, or both may be immobilized on a solid support. The solid support may be in the form of a bead, box, column, cylinder, disc, dish (e.g., glass dish, PETRI dish), fibre, film, filter, microtiter plate (e.g., 96-well microtiter plate), multi-bladed stick, net, pellet, plate, ring, rod, roll, sheet, slide, stick, tray, tube, or vial. The solid phase support can be a singular discrete body (e.g., a single tube, a single bead), any number of a plurality of substrate bodies (e.g., a rack of 10 tubes, several beads), or combinations thereof (e.g., a tray comprises a plurality of microtiter plates, a column filled with beads, a microtiter plate filed with beads). The solid support may be a surface of a bead, tube, tank, tray, dish, a plate, a flask, or a bag. The solid support may be an array. The solid support may be a bag.

In an embodiment, the introduction of a nucleic acid into the T cell may comprise transfecting a naked DNA comprising the nucleic acid. The introduction of a nucleic acid into the T cell may comprise transducing a viral vector comprising the nucleic acid. The viral vector may be a retroviral vector, an adenoviral vector, an adeno-associated viral vector, or a lentiviral vector. The nucleic acid may encode a recombinant protein. The recombinant protein may be a chimeric antigen receptor (CAR), a T cell receptor (TCR), a cytokine, an antibody, or a bi-specific binding molecule. The nucleic acid may encode a T cell receptor (TCR).

In one embodiment, the expansion of the T cells may be in the presence of a cytokine. The cytokine may be interferon alpha (IFN-α), interleukin-2 (IL-2), interleukin-4 (IL-4), interleukin-7 (IL-7), interleukin-9 (IL-9), interleukin-12 (IL-12), interleukin-15 (IL-15), interleukin-21 (IL-21), or a combination thereof.

In an embodiment, the cytokine may be interferon alpha (IFN-α), interleukin-2 (IL-2), interleukin-4 (IL-4), interleukin-7 (IL-7), interleukin-9 (IL-9), interleukin-12 (IL-12), interleukin-15 (IL-15), interleukin-21 (IL-21), or a combination thereof and the cytokine may be present in an amount at about 1 ng/mL and 500 ng/mL. The cytokine may be present in an amount of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 ng/mL. The cytokine may be present in an amount between about 1 ng/mL and 100 ng/mL, about 100 ng/mL and 200 ng/mL, about 100 ng/mL and 500 ng/mL, about 250 ng/mL and 400 ng/mL, about 10 ng/mL and 100 ng/mL, or about 150 ng/mL and 350 ng/mL.

In one embodiment, the cytokine may comprise a combination of IL-7 and IL-15.

In one embodiment, the concentration of IL-7 may be from about 1 ng/ml to 100 ng/ml, about 1 ng/ml to 90 ng/ml, about 1 ng/ml to 80 ng/ml, about 1 ng/ml to 70 ng/ml, about 1 ng/ml to 60 ng/ml, about 1 ng/ml to 50 ng/ml, about 1 ng/ml to 40 ng/ml, about 1 ng/ml to 30 ng/ml, about 1 ng/ml to 20 ng/ml, about 1 ng/ml to 15 ng/ml, or about 1 ng/ml to 10 ng/ml.

In one embodiment, the IL-7 may be present in an amount at about 1 ng/mL and 500 ng/mL. The cytokine may be present in an amount of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 ng/mL. The cytokine may be present in an amount between about 1 ng/mL and 100 ng/mL, about 100 ng/mL and 200 ng/mL, about 100 ng/mL and 500 ng/mL, about 250 ng/mL and 400 ng/mL, about 10 ng/mL and 100 ng/mL, or about 150 ng/mL and 350 ng/mL.

In one embodiment, the concentration of IL-15 may be from about 5 ng/ml to 500 ng/ml, about 5 ng/ml to 400 ng/ml, about 5 ng/ml to 300 ng/ml, about 5 ng/ml to 200 ng/ml, about 5 ng/ml to 150 ng/ml, about 5 ng/ml to 100 ng/ml, about 10 ng/ml to 100 ng/ml, about 20 ng/ml to 100 ng/ml, about 30 ng/ml to 100 ng/ml, about 40 ng/ml to 100 ng/ml, or about 50 ng/ml to 100 ng/ml.

In an embodiment, the IL-15 may be present in an amount at about 1 ng/mL and 500 ng/mL. The cytokine may be present in an amount of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 ng/mL. The cytokine may be present in an amount between about 1 ng/mL and 100 ng/mL, about 100 ng/mL and 200 ng/mL, about 100 ng/mL and 500 ng/mL, about 250 ng/mL and 400 ng/mL, about 10 ng/mL and 100 ng/mL, or about 150 ng/mL and 350 ng/mL.

In one embodiment, the step (a) through the step (e) may be performed in a closed system.

In an embodiment, the number of the harvested T cells produced by the methods described herein may be from about 1×10⁹ to about 1×10¹³, about 1×10⁹ to about 5×10¹², about 1×10⁹ to about 1×10¹², about 1×10⁹ to about 5×10¹¹, about 1×10⁹ to about 1×10¹¹, about 1×10⁹ to about 5×10¹⁰, about 1×10⁹ to about 1×10¹⁰, about 2×10⁹ to about 1×10¹⁰, about 3×10⁹ to about 1×10¹⁰, about 4×10⁹ to about 1×10¹⁰, about 5×10⁹ to about 1×10¹⁰, about 6×10⁹ to about 1×10¹⁰, about 7×10⁹ to about 1×10¹⁰, about 8×10⁹ to about 1×10¹⁰, or about 9×10⁹ to about 1×10¹⁰ cells.

In an embodiment, the number of the harvested T cells produced by the methods described herein may be about 1×10⁹ cells, 2×10⁹ cells, 3×10⁹ cells, 4×10⁹ cells, 5×10⁹ cells, 6×10⁹ cells, 7×10⁹ cells, 8×10⁹ cells, 9×10⁹ cells, 1×10¹⁰ cells, 1×10¹⁰ cells, 2×10¹⁰ cells, 3×10¹⁰ cells, 4×10¹⁰ cells, 5×10¹⁰ cells, 6×10¹⁰ cells, 7×10¹⁰ cells, 8×10¹⁰ cells, 9×10¹⁰ cells, 1×10¹¹ cells, 2×10¹¹ cells, 3×10¹¹ cells, 4×10¹¹ cells, 5×10¹¹ cells, 6×10¹¹ cells, 7×10¹¹ cells, 8×10¹¹ cells, 9×10¹¹ cells, 1×10¹² cells, 2×10¹² cells, 3×10¹² cells, 4×10¹² cells, 5×10¹² cells, 6×10¹² cells, 7×10¹² cells, 8×10¹² cells, 9×10¹² cells, 1×10¹³ cells, 2×10¹³ cells, 3×10¹³ cells, 4×10¹³ cells, 5×10¹³ cells, 6×10¹³ cells, 7×10¹³ cells, 8×10¹³ cells, 9×10¹³ cells, or 1×10¹⁴ cells.

In one embodiment, a population of genetically modified T cells may be produced by the methods described herein.

In an embodiment, a method of treating a patient who has cancer may comprise administering to the patient a composition comprising a population of genetically modified T cells described herein, wherein the genetically modified T cells kill cancer cells that present a peptide in a complex with an MHC molecule on the surface, wherein the peptide is selected from SEQ ID NO: 1-160, and the cancer is selected from the group consisting of hepatocellular carcinoma (HCC), colorectal carcinoma (CRC), glioblastoma (GB), gastric cancer (GC), esophageal cancer, non-small cell lung cancer (NSCLC), pancreatic cancer (PC), renal cell carcinoma (RCC), benign prostate hyperplasia (BPH), prostate cancer (PCA), ovarian cancer (OC), melanoma, breast cancer, chronic lymphocytic leukemia (CLL), Merkel cell carcinoma (MCC), small cell lung cancer (SCLC), Non-Hodgkin lymphoma (NHL), acute myeloid leukemia (AML), gallbladder cancer and cholangiocarcinoma (GBC, CCC), urinary bladder cancer (UBC), acute lymphocytic leukemia (ALL), and uterine cancer (UEC). The MHC molecule may be MHC Class I.

In one embodiment, the composition may further comprise an adjuvant.

In one embodiment, the adjuvant may be an anti-CD40 antibody, imiquimod, resiquimod, GM-CSF, cyclophosphamide, sunitinib, bevacizumab, atezolizuma, interferon-alpha, interferon-beta, CpG oligonucleotides and derivatives, poly-(I:C) and derivatives, RNA, sildenafil, particulate formulations with poly(lactide co-glycolide) (PLG), virosomes, interleukin-1 (IL-1), interleukin-2 (IL-2), interleukin-4 (IL-4), interleukin-7 (IL-7), interleukin-12 (IL-12), interleukin-13 (IL-13), interleukin-15 (IL-15), interleukin-21 (IL-21), interleukin-23 (IL-23), or a combination thereof.

In one embodiment, a method of eliciting an immune response in a patient who has cancer may comprise administering to the patient a composition comprising the population of genetically modified T cells described herein, wherein the genetically modified T cells kill cancer cells that present a peptide in a complex with an MHC molecule on the surface, wherein the peptide is selected from SEQ ID NO: 1-160, wherein the cancer is selected from the group consisting of hepatocellular carcinoma (HCC), colorectal carcinoma (CRC), glioblastoma (GB), gastric cancer (GC), esophageal cancer, non-small cell lung cancer (NSCLC), pancreatic cancer (PC), renal cell carcinoma (RCC), benign prostate hyperplasia (BPH), prostate cancer (PCA), ovarian cancer (OC), melanoma, breast cancer, chronic lymphocytic leukemia (CLL), Merkel cell carcinoma (MCC), small cell lung cancer (SCLC), Non-Hodgkin lymphoma (NHL), acute myeloid leukemia (AML), gallbladder cancer and cholangiocarcinoma (GBC, CCC), urinary bladder cancer (UBC), acute lymphocytic leukemia (ALL), and uterine cancer (UEC).

In one embodiment, the introducing a nucleic acid into the activated non-adherent cell population may be performed with or without serum.

In one embodiment, the introducing a nucleic acid into the activated non-adherent cell population may be performed without serum.

In an embodiment, a method for producing an engineered T cell population may include obtaining a cell population comprising a monocyte and a T cell, resting the obtained cell population on a surface, adhering the monocyte to the surface, retaining a non-adherent cell population, activating the non-adherent cell population, introducing a nucleic acid into the activated non-adherent cell population to obtain a transformed T cell, and expanding the transformed T cell to obtain the engineered T cell population.

In one embodiment, the cell population may contain peripheral blood mononuclear cells (PMBC).

In one embodiment, the monocyte may include a CD14+ cell.

In one embodiment, the T cell may include a αβ T cell and/or a γδ T cell.

In one embodiment, the T cell may include a CD8+ T cell and/or a CD4+ T cell.

In one embodiment, the resting may be performed for 2-8 hours.

In one embodiment, the resting may be performed at a seeding density of 0.1×10⁶/cm2-2×10⁶/cm2.

In one embodiment, the surface may contain a plastic or a glass.

In one embodiment, the plastic may contain polystyrene or polycarbonate.

In one embodiment, the surface may contain a plurality of cell growth areas.

In one embodiment, the plurality of cell growth areas may be configured in the form of a plurality of stacks.

In one embodiment, the plurality of stacks may contain at least 2, at least 3, at least 4, at least 5, at least 6, at least 7, at least 8, at least 9, at least 10, at least 15, at least 20, at least 25, at least 30, at least 35, at least 40, at least 45, at least 50 stacks, at least 60 stacks, at least 70 stacks, at least 80 stacks, at least 90 stacks, or at least 100 stacks.

In one embodiment, the plurality of cell growth areas may contain at least 400 cm², at least 500 cm², at least 600 cm², at least 700 cm², at least 800 cm², at least 900 cm², at least 1,000 cm², at least 2,000 cm², at least 3,000 cm², at least 4,000 cm², at least 5,000 cm², at least 6,000 cm², at least 7,000 cm², at least 8,000 cm², at least 9,000 cm², at least 10,000 cm², at least 20,000 cm², at least 30,000 cm², at least 40,000 cm², or at least 50,000 cm².

In one embodiment, the activating may be performed in the presence of an anti-CD3 antibody and an anti-CD28 antibody.

In one embodiment, the nucleic acid may encode a recombinant protein.

In one embodiment, the recombinant protein may be a chimeric antigen receptor (CAR), a T cell receptor (TCR), a cytokine, an antibody, or a bi-specific binding molecule.

In one embodiment, the recombinant protein may be a TCR.

In one embodiment, the TCR may bind a peptide in a complex with an MHC molecule.

In one embodiment, the peptide may be one selected from SEQ ID NOS: 1-161.

In one embodiment, the MHC molecule may be a class I MHC molecule.

In one embodiment, the cell population may contain at least 25% monocyte.

In one embodiment, the non-adherent cell population may be a monocyte-deprived cell population.

In one embodiment, the cell population may further contain a myeloid derived suppressor cell (MDSC).

In one embodiment, the MDSC may be a CD124+/CD14+/CD3−/CD19−/CD56− cell, a CD124+/CD15+/CD3−/CD19−/CD56− cell, and/or a CD14−/CD15−/CD33hiCD3−/CD19−/CD56− cell.

In one embodiment, the MDSC may be adhered to the surface.

In an embodiment, a composition may contain the engineered T cell population produced by the method of the present disclosure.

In one embodiment, the nucleic acid may further encode a CD8αβ heterodimer or a CD8α homodimer.

In one embodiment, the CD8α may have the amino acid sequence selected from SEQ ID NO: 163-166 and the CD8β may have the amino acid sequence selected from SEQ ID NO: 167-173.

In one embodiment, the nucleic acid may further contain a woodchuck hepatitis virus posttranscriptional responsive element (WPRE) comprising the nucleotide sequence selected from SEQ ID NO: 174-176.

### BRIEF DESCRIPTION OF THE DRAWINGS

FIG. 1 depicts the correlation of monocytes (%) with activation markers. N=16 patients.

FIGS. 2A-2C depict grouped fold expansion for Grex PBMCs NT (non-transduced; refers to PBMCs rested in Grex and are not transduced with the TCR), Grex PMBCs TCR (refers to PBMCs rested in Grex and are transduced with the TCR), which act as controls, versus populations with 10%, 30%, 60%, or 80% monocyte enriched PBMC populations (2A) (expansion based on total number of cells), grouped TCR+ frequency (%) (2B), and grouped absolute number of TCR+ cells (2C).

FIGS. 3A-3C depict the total cell count (3A), percent recovery (3B), and percent viability (3C) of cells, pre-rest and post-rest with different percentages of monocytes including 60% monocytes. Briefly, healthy donor material (PBMCS) was either rested in a Grex or cell stack (CS)/flask for 4- or 2-hours, respectively. The conditions listed as “Grex 60% mono or CS/Flask 60% mono” refer to starting material (PBMC) that was artificially seeded with increased monocytes such that the frequency of monocytes in that starting material was 60%. This was done to simulate starting material with high monocyte content (typically above 20%). Pre-rest refers to total cell counts (performed via a Cellometer) taken before the rest and post-rest are counts taken after the rest.

FIGS. 4A-4B depict the fold expansion of NT, Grex, CS/Flask, Grex 60% monocytes, and CS/Flask 60% monocytes (4A) and absolute TCR+ cell counts (4B). Briefly, healthy donor material (PBMCS) rested in a Grex or cell stack (CS)/flask for 4- or 2-hours, respectively, which then went through manufacturing process (activation/transduction/expansion) and was harvested on day 7. FIG. 4A depicts the fold expansion of total cells from the time of transduction to harvest. 8×10⁶ cells per condition (per donor) were transduced and [harvest count]/8×10⁶ will yield the total fold expansion. FIG. 4B shows absolute TCR+ cell counts referring to the total number of CD3+CD8+ cells that are tetramer positive.

Grouped data, n=4, 3 donors rests in CS and 1 in flask; Day harvest metrics.

FIGS. 5A-5B depict the residual populations, pre- and post-rest. The percentage of positive cells, B cells, γδ T cells, monocytes, natural killer cells, and T-cells were measured (gated on live cells) (5A) and MDSC1 (CD124+/CD14+/CD3−/CD19−/CD56−), MDSC2 (CD124+/CD15+/CD3−/CD19−/CD56−), and MDSC3 (CD14−/CD15−/CD33hiCD3−/CD19−/CD56−) (5B). [

FIG. 6 depicts increased activation of CD8+ T cells rested with cellstacks. The cells were gated on live CD8+ T cells. CS=monocyte depletion using plastic adherence and G-Rex=control.

FIGS. 7A-7B depict that immune check point inhibitor marker expression was reduced in products generated with the monocyte depletion methods described herein (7A). Also, a decrease in CD39+/CD69+ cells and an increase in CD39−/CD69− cells (7B).

FIG. 8 depicts patient data (n=13 and n=4 optimized conditions #10-#13) showed an improved yield in the TCR+CD8+ T cells at harvest. Grex=control and CS/Flask=monocyte depleted.

FIG. 9A shows higher fold expansion from transduction to harvest in T cell products prepared by monocyte depletion using plastic adherence (CS) than that using G-Rex.

FIG. 9B shows higher % CD8+ cells in T cell products prepared by monocyte depletion using plastic adherence (CS) than that using G-Rex.

FIG. 10A shows number of TCR+CD8+ T cells prepared by monocyte depletion using plastic adherence (CS) is higher than that using G-Rex in patents A, B, and D, whose % monocytes are higher than % CD3+ cells.

FIG. 10B shows the average number of TCR+CD8+ T cells prepared by CS are higher than that using G-Rex in patents A-D.

FIG. 10C shows the average % TCR+CD8+ T cells prepared by CS are higher than that using G-Rex in patents A-D.

FIG. 11A shows that TCR+CD8+ T cells prepared by monocyte depletion using plastic adherence (CS) contained higher % of CD45RA+ and CD28+ cells and lower % of CD45RO+ cells than that prepared by using G-Rex.

FIG. 11B shows that TCR+CD8+ T cells prepared by CS contained higher % of naïve T cells than that prepared by using G-Rex.

FIG. 12A shows that tumor killing activity of TCR+CD8+ T cells prepared by CS is comparable to that prepared by using Grex.

FIG. 12B shows no significant difference in cell killing between TCR+CD8+ T cells prepared by CS and Grex

FIG. 13A shows that higher % monocyte present in all patients at pre-resting correlates with higher fold change of CS over Grex.

FIG. 13B shows that higher % monocyte present in only patients with optimized conditions at pre-resting correlates with higher fold change of CS over Grex.

FIG. 13C shows that higher % monocyte present in only patients with unoptimized conditions were measured at pre-resting correlates with higher fold change of CS over Grex.

FIG. 13D shows that patients with high monocyte frequency (>25% monocytes) appear most benefitted by depleting them using optimized rest & expansion conditions.

FIG. 14A shows that 2 hours rest led to more efficient monocyte depletion than 4 hr rest and that both 0.5 and 0.8×10⁶/cm′ seeding densities led to comparable depletion efficiency.

FIG. 14B shows that yield of TCR+CD8+ cells in Cellstack (CS)-rested cells appear comparable to flasks. Both 0.5 and 0.8×10⁶/cm′ seeding densities appear comparable in transduced cell yields.

FIG. 15 shows that Corning® CellSTACK® performed better for monocyte depletion as compared with that obtained from other manufacturers.

FIG. 16 shows that, at post-rest, % monocytes were comparable among T cells prepared by monocyte depletion using cellstacks with increasing cell growth areas.

FIG. 17 shows that, at post-rest, % MDSCs were comparable among T cells prepared by monocyte depletion using cellstacks with increasing cell growth areas.

FIG. 18A shows the effect of monocyte depletion on frequency of CD8 T cells in accordance with one embodiment of the present disclosure.

FIG. 18B shows the effect of monocyte depletion on transduction efficiency in accordance with one embodiment of the present disclosure.

FIG. 18C shows the effect of monocyte depletion on fold expansion in accordance with one embodiment of the present disclosure.

FIG. 19A shows the effect of serum free transduction on frequency of CD8 T cells in accordance with one embodiment of the present disclosure.

FIG. 19B shows the effect of serum free transduction on transduction efficiency in accordance with one embodiment of the present disclosure.

FIG. 19C shows the effect of serum free transduction on fold expansion in accordance with one embodiment of the present disclosure.

### DETAILED DESCRIPTION OF PREFERRED EMBODIMENTS

### Adoptive T-Cell Therapy

Adoptive T-cell therapy using genetically modified T cells is an attractive strategy in various clinical settings. Ho et al. Cancer Cell (2003) 3: 431-437. A short, e.g., 6-day, manufacturing process for producing genetically modified T cell products expressing recombinant proteins, such as chimeric antigen receptors (CARs), T cell receptors (TCRs), cytokines, antibodies, and bi-specific binding molecules, yields products with less differentiated memory phenotype as compared to the longer, e.g., 8-10 day, processes. Although a short manufacturing process may be an asset to the adoptive T-cell therapy, the total cell number of functionally transduced T-cells may be compromised by the short manufacturing process, especially when higher T-cell doses are preferred for infusion in cancer patients. To meet the need for higher doses, various strategies can be used to increase the total yield of functionally transduced cells. These may include scaling-up the whole process, enhancing the transduction efficiency or starting from CD8+ selected T cells as opposed to the bulk PBMC for a CD8 dependent TCR. Although further scale-up of the manufacturing process may be achievable, it may be, however, more expensive, more lengthy, and may impact manufacturing capacity.

The pool of lymphocytes, from which CD8+ T cells, for example, for adoptive immunotherapy can be derived, may contain naive and long-lived antigen experienced memory T cells (T_{M}). T_{M} can be divided further into subsets of central memory (T_{CM}) and effector memory (T_{EM}) cells that differ in phenotype, homing properties and functions. CD8+T_{CM} express CD62L and CCR7, which promote migration into lymph nodes, and proliferate rapidly if re-exposed to antigen. CD8+T_{EM} lack CD62L enabling migration to peripheral tissues, and exhibit immediate effector function. In response to antigen stimulation, CD8+T_{CM} and T_{EM} both differentiate into cytolytic effector T cells (T_{E}) that express a high level of granzymes and perforin, but are short-lived. Thus, the poor survival of T cells in clinical immunotherapy trials may simply result from their differentiation during in vitro culture to T_{E} that are destined to die.

To address this issue, the inventors used CD8+ selected T cells as starting material to produce genetically modified T cell products expressing recombinant proteins, e.g., CARs, TCRs, cytokines, antibodies, and bi-specific binding molecules, which yield a greater number of genetically modified T cell products, e.g., CAR- or TCR-transformed T cell products than in large- or GMP-scale that manufactured using PBMC as starting materials, while maintaining comparable functionality of genetically modified T cell products manufactured by either process. This leads to a surprising increase in the yield of desired T cells without expensive scale-up, replication costs, or a lengthy processing time (e.g., greater than 7 days). The inventors also found that T-cells which were not CD8+ may also be used with excellent yields. Further the inventors also included a step where adherent cells, e.g., monocytes, were depleted from the cell population. In an embodiment, the inventors also used (1) a closed system, (2) CD8+ selected T cells as starting material, and (3) activation with anti-CD3/anti-CD28 antibodies followed by transduction with viral vectors, e.g., lentiviral vectors, expressing recombinant proteins to produce genetically modified T cell products expressing recombinant proteins, as above.

The inventors found that several manufacturing attempts resulted in lower-than-expected yields of usable T-cells for therapy. As a result of extensive study of failed manufacturing attempts, the inventors found that inhibitory cells, for example monocytes, showed the strongest correlation with lower cell yields. yields. The art did not offer any clear guidance on how to identify the underlying reason for the lower cell. For example, U.S. Patent Application Publication No. 2021/0046159 suggested that monocyte depletion had a negative impact on the T-cell expansion.

### Definitions

“Activation” as used herein refers broadly to the state of a T cell that has been sufficiently stimulated to induce detectable cellular proliferation. Activation can also be associated with induced cytokine production, and detectable effector functions. The term “activated T cells” refers to, among other things, T cells that are proliferating.

“Antibodies” and “immunoglobulin” as used herein refer broadly to antibodies or immunoglobulins of any isotype, fragments of antibodies, which retain specific binding to antigen, including, but not limited to, Fab, Fab′, Fab′-SH, (Fab′)₂ Fv, scFv, divalent scFv, and Fd fragments, chimeric antibodies, humanized antibodies, single-chain antibodies, and fusion proteins including an antigen-specific targeting region of an antibody and a non-antibody protein.

“Bispecific binding molecule” and “bispecific antigen binding molecule,” as used herein refer broadly to antigen-binding proteins are able of binding to two different antigens simultaneously, e.g., bispecific antibodies. For example, unlike conventional antibodies, the bispecific antigen binding molecule of the present disclosure may comprise at least 6 CDRs from a TCR. In an embodiment, the antigen binding proteins of the present disclosure, unlike conventional antibodies, may comprise at least one variable alpha domain and at least one variable beta domain from a TCR.

“Chimeric antigen receptor” or “CAR” or “CARs” as used herein refers broadly to genetically modified receptors, which graft an antigen specificity onto cells, for example T cells, NK cells, macrophages, and stem cells. CARs can include at least one antigen-specific targeting region (ASTR), a hinge or stalk domain, a transmembrane domain (TM), one or more co-stimulatory domains (CSDs), and an intracellular activating domain (IAD). In certain embodiments, the CSD is optional. In another embodiment, the CAR is a bispecific CAR, which is specific to two different antigens or epitopes. After the ASTR binds specifically to a target antigen, the IAD activates intracellular signaling. For example, the IAD can redirect T cell specificity and reactivity toward a selected target in a non-MHC-restricted manner, exploiting the antigen-binding properties of antibodies. The non-MHC-restricted antigen recognition gives T cells expressing the CAR the ability to recognize an antigen independent of antigen processing, thus bypassing a major mechanism of tumor escape. Moreover, when expressed in T cells, CARs advantageously do not dimerize with endogenous T cell receptor (TCR) alpha and beta chains.

“Cytotoxic T lymphocyte” (CTL) as used herein refers broadly to a T lymphocyte that kills cancer cells, cells that are infected particularly with viruses), or cells that are damaged in other ways. The CTL may express CD8 on the surface thereof (e.g., a CD8+ T cell). Such cells may be preferably “memory” T cells (T_{M} cells) that are antigen-experienced.

“Donors” as used herein refers broadly human subjects that donated blood.

“Effective amount”, “therapeutically effective amount”, or “efficacious amount” as used herein refers broadly to the amount of an agent, or combined amounts of two agents, that, when administered to a mammal or other subject for treating a disease, is sufficient to affect such treatment for the disease. The “therapeutically effective amount” will vary depending on the agent(s), the disease and its severity and the age, weight, etc., of the subject to be treated.

“Genetically modified” as used herein refers broadly to methods to introduce exogenous nucleic acids into a cell, whether or not the exogenous nucleic acids are integrated into the genome of the cell.

“Genetically modified cell” as used herein refers broadly to cells that contain exogenous nucleic acids whether or not the exogenous nucleic acids are integrated into the genome of the cell.

“Immune cells” as used herein refers broadly to white blood cells (leukocytes) derived from hematopoietic stem cells (HSC) produced in the bone marrow “Immune cells” include, without limitation, lymphocytes (T cells, B cells, natural killer (NK) (CD3-CD56+) cells) and myeloid-derived cells (neutrophil, eosinophil, basophil, monocyte, macrophage, dendritic cells). “T cells” include all types of immune cells expressing CD3 including T-helper cells (CD4+ cells), cytotoxic T-cells (CD8+ cells), T-regulatory cells (Treg) and gamma-delta T cells, and NK T cells (CD3+ and CD56+). A skilled artisan will understand T cells and/or NK cells, as used throughout the disclosure, can include only T cells, only NK cells, or both T cells and NK cells. In certain illustrative embodiments and aspects provided herein, T cells are activated and transduced. Furthermore, T cells are provided in certain illustrative composition embodiments and aspects provided herein. A “cytotoxic cell” includes CD8+ T cells, natural-killer (NK) cells, NK-T cells, γδ T cells, and neutrophils, which are cells capable of mediating cytotoxicity responses.

Myeloid derived suppressor cells (MDSCS)” referred to herein may include heterogeneous population of myeloid cells that usually exert a suppressive or negative effect on innate or adaptive immune cells (at least in the context of cancer control by the immune system or in cancer immunotherapy, these cells are considered detrimental to positive outcomes). In our experiments, we specifically looked at the MDSC1, MDSC2, and MDSC7 subsets.

“Individual,” “subject,” “host,” and “patient,” as used interchangeably herein, refer broadly to a mammal, including, but not limited to, humans, murines (e.g., rats, mice), lagomorphs (e.g., rabbits), non-human primates, canines, felines, and ungulates (e.g., equines, bovines, ovines, porcines, caprines).

“Peripheral blood mononuclear cells” or “PBMCs” as used herein refers broadly to any peripheral blood cell having a round nucleus. PBMCs include lymphocytes, such as T cells, B cells, and NK cells, and monocytes.

“Polynucleotide” and “nucleic acid”, as used interchangeably herein, refer broadly to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer including purine and pyrimidine bases or other natural, chemically or biochemically modified, non-natural, or derivatized nucleotide bases.

“T cell” or “T lymphocyte” are art-recognized terms and include thymocytes, naïve T lymphocytes, immature T lymphocytes, mature T lymphocytes, resting T lymphocytes, or activated T lymphocytes. Illustrative populations of T cells suitable for use in particular embodiments include, but are not limited to, helper T cells (HTL; CD4+ T cell), a cytotoxic T cell (CTL; CD8+ T cell), CD4+CD8+ T cell, CD4−CD8− T cell, or any other subset of T cells. Other illustrative populations of T cells suitable for use in particular embodiments include, but are not limited to, T cells expressing one or more of the following markers: CD3, CD4, CD8, CD27, CD28, CD45RA, CD45RO, CD62L, CD127, CD197, and HLA-DR and if desired, can be further isolated by positive or negative selection techniques.

“T-cell receptor (TCR)” as used herein refers broadly to a protein receptor on T cells that is composed of a heterodimer of an alpha (α) and beta (β) chain, although in some cells the TCR consists of gamma and delta (γ/δ) chains. The TCR may be modified on any cell comprising a TCR, including a helper T cell, a cytotoxic T cell, a memory T cell, regulatory T cell, natural killer T cell, or a gamma delta T cell.

The TCR is generally found on the surface of T lymphocytes (or T cells) that is generally responsible for recognizing antigens bound to major histocompatibility complex (MHC) molecules. It is a heterodimer consisting of an alpha and beta chain in 95% of T cells, while 5% of T cells have TCRs consisting of gamma and delta chains. Engagement of the TCR with antigen and MHC results in activation of its T lymphocyte through a series of biochemical events mediated by associated enzymes, co-receptors, and specialized accessory molecules. In immunology, the CD3 antigen (CD stands for cluster of differentiation) is a protein complex composed of four distinct chains (CD3-γ, CD3δ, and two times CD3ε) in mammals that associate with molecules known as the T-cell receptor (TCR) and the ζ-chain to generate an activation signal in T lymphocytes. The TCR, ζ-chain, and CD3 molecules together comprise the TCR complex. The CD3-γ, CD3δ, and CD3ε chains are highly related cell surface proteins of the immunoglobulin superfamily containing a single extracellular immunoglobulin domain. The transmembrane region of the CD3 chains is negatively charged, a characteristic that allows these chains to associate with the positively charged TCR chains (TCRα and TCRβ). The intracellular tails of the CD3 molecules contain a single conserved motif known as an immunoreceptor tyrosine-based activation motif or ITAM for short, which is essential for the signalling capacity of the TCR.

“Treatment,” “treating,” and the like, as used herein refer broadly to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, e.g., arresting its development; and (c) relieving the disease, e.g., causing regression of the disease.

### Peripheral Blood Mononuclear Cells (PMBC)

Peripheral Blood Mononuclear Cells (PMBC) may be obtained from a patient, optionally a healthy patient. The PMBC may be frozen for later use. After the PMBC population is obtained, optionally thawed, the adherent cells may be depleted from the PMBC population. The adherent cells may be depleted by a method comprises resting the PMBC in a vessel for a time sufficient for portion of the cells to adhere to the vessel and removing the non-adherent cells. The adherent cells may comprise monocytes, which show a strong negative correlation with total cell yields.

The time sufficient for portion of the cells to adhere to the vessel may be between about 1 and 10 hours. The time may be about 1, 2, 3, 4, 5, 6, 7, 8, 9, or 10 hours. The time may be between about 1-6 hours, 2-4 hours, 1-4 hours, 3-6 hours, 4-10 hours, or 2-3 hours.

The cell culture vessel may be plastic or glass. The plastic may be polysterene or polycarbonate. The cell culture vessel may be treated with a coating.

The PMBC may be seeded in the vessel at a density of cells between about 0.1 and 2.0 million cells/cm². The density of the cells may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 million cells/cm². The density of the cells may be about 0.3, 0.5, 0.8, or 1.2 million cells/cm². The density of the cells may be between about 0.1 to 2.0 million cells/cm², 0.3 to 1.0 million cells/cm², 0.5 to 0.8 million cells/cm², 0.5 to 1.0 million cells/cm², 0.3 to 1.5 million cells/cm², 0.8 to 2.0 million cells/cm², or 1.0 to 2.0 million cells/cm². The cell culture vessel may be a flask, dish, bag, cellstack, or an assemblage thereof. The cell culture vessel may comprise a plurality of flasks, dishes, bags, cellstacks, or an assemblage thereof.

The PMBC cells may be thawed (if frozen), rested, and then activated as described herein. The conditions at rest may comprise seeding the cells at about 5×10⁶ cells/mL and resting for 4 hours, seeding the cells at about 5×10⁶ cells/mL and resting for 2 hours, seeding the cells at about 0.8×10⁶ cells/cm² and resting for 2 hours, seeding the cells at about 0.5×10⁶ cells/cm² and resting for 2 hours, seeding the cells at about 0.8×10⁶ cells/cm² and resting for 4 hours, or seeding the cells at about 0.5×10⁶ cells/cm² and resting for 4 hours. After the rest, the adherent cells adhere to the plastic vessel. Non-adherent cells may be harvested, for example, by gently rotating the vessel 4-5 times and pipetting out the media containing the non-adherent cells.

### Monocyte Depletion

If desired or necessary, monocyte populations, e.g., CD14+ cells, may be depleted from blood preparations prior to ex vivo expansion by a variety of methodologies, including plastic, glass, anti-CD14 coated beads or columns, or utilization of the phagocytotic activity of these cells to facilitate removal. In certain embodiments, plastic-adherent CD14+CD1a− monocytes may be purified from adult human blood, e.g., PBMC. Monocytes may be then further purified or removed using plastic adherence as described by Zhou et al. (J. Immunology 154:3821-3835, 1995; the content of which is hereby incorporated by reference in its entirety). In one embodiment, the paramagnetic particles of a size may be sufficient to be engulfed by phagocytotic monocytes. In certain embodiments, the paramagnetic particles are commercially available beads, for example, those produced by Dynal AS under the trade name Dynabeads™. Exemplary Dynabeads™ in this regard are M-280, M-450, and M-500. In one aspect, other non-specific cells may be removed by coating the paramagnetic particles with “irrelevant” proteins (e.g., serum proteins or antibodies). Irrelevant proteins and antibodies include those proteins and antibodies or fragments thereof that do not specifically target the T cells to be expanded. In certain embodiments the irrelevant beads may include beads coated with sheep anti-mouse antibodies, goat anti-mouse antibodies, and human serum albumin. In brief, such depletion of monocytes may be performed by preincubating PBMC isolated from whole blood or apheresed peripheral blood with one or more varieties of irrelevant or non-antibody coupled paramagnetic particles at any amount that may allow for removal of monocytes (approximately a 20:1 bead:cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after said depletion.

In one embodiment, monocyte depletion may increase frequency of CD8+ T cells, e.g., CD8+CD3+ T cells, in T cell products by from about 5% to about 60%, from about 5% to about 50%, from about 5% to about 45%, from about 5% to about 40%, from about 5% to about 35%, from about 5% to about 30%, from about 5% to about 25%, from about 5% to about 20%, from about 5% to about 15%, from about 5% to about 10%, from about 10% to about 50%, from about 10% to about 40%, from about 10% to about 30%, from about 10% to about 20%, from about 10% to about 15%, about 5%, about 10%, about 15%, about 20%, about 25%, about 30%, about 35%, about 40%, about 45%, about 50%, about 55%, or about 60%, as compared with that without monocyte depletion.

In one embodiment, monocyte depletion may increase transduction efficiency of exogenous TCR in CD8, e.g., peptide/MHC Dextramer (Dex)+CD8+ T cells, by from about 5% to about 60%, from about 5% to about 50%, from about 5% to about 45%, from about 5% to about 40%, from about 5% to about 35%, from about 5% to about 30%, from about 5% to about 25%, from about 5% to about 20%, from about 5% to about 15%, from about 5% to about 10%, from about 10% to about 50%, from about 10% to about 40%, from about 10% to about 30%, from about 10% to about 20%, from about 10% to about 15%, about 5%, about 10%, about 15%, about 20%, about 25%, about 30%, about 35%, about 40%, about 45%, about 50%, about 55%, or about 60%, as compared with that without monocyte depletion.

### Isolation of T Cells

T cells may be isolated from preparations of peripheral blood mononuclear cells (PBMCs) by positive or negative selection, or both.

After depletion of non-CD8+ cells, e.g., CD4+ T cells, monocytes, neutrophils, eosinophils, B cells, stem cells, dendritic cells, NK cells, granulocytes, γ/δ T cells, or erythroid cells, The T cells are collected, and, optionally, stored, until used in a method described herein for the production of genetically modified T cells.

### Isolation of CD8+ T Cells

Since CD8+ T cells have relatively simple functions as compared with other cells, such as dendritic cells, CD4+ T cells, and NK cells, it is less likely for CD8+ T cells to cause unexpected side effects during anticancer immunotherapy. Generally, antigen-specific CD8+ T cells may be isolated by using MHC class I/peptide multimer, which, however, may stimulate a T cell receptor (TCR). As such, this method may have some drawbacks including high cell death rate caused by cell apoptosis after cell isolation and a long period of culturing time required to produce sufficient amounts of antigen-specific CD8+ T cells.

CD8+ T cells may be isolated from preparations of peripheral blood mononuclear cells (PBMCs) by positive or negative selection, or both. Positive selection may result in a highly-purified population of CD8+ cells. Negative selection, e.g., depleting CD4+ cells, while resulting in sufficient numbers of CD8+ cells, may have low levels of contaminating non-CD8+ populations remaining after the selection procedure. CD8+ T cells may be isolated from preparations of PBMCs using, e.g., anti-CD8 antibodies, which may have high affinity for CD8+ cells, may not activate the cells during the selection process, and may be capable of being easily eluted from the cells. Anti-CD8 antibodies are known in the art and are commercially available.

In another embodiment of the present disclosure, CD8+ cells may be CD8+CD62L+ T cells, which may be isolated using a two-step procedure. After depletion of non-CD8+ cells, e.g., CD4+ T cells, monocytes, neutrophils, eosinophils, B cells, stem cells, dendritic cells, NK cells, granulocytes, γ/δ T cells, or erythroid cells, which may be labeled by using a cocktail of biotin-conjugated antibodies that may contain antibodies against, e.g., CD4, CD15, CD16, CD19, CD34, CD36, CD56, CD123, TCRγ/δ, and/or CD235a (Glycophorin A), the CD8+CD62L+ T cells may be positively isolated using CD62L microbeads. The magnetically labeled CD8+CD62L+ T cells may be retained within the column, e.g., MACS column (Miltenyi Biotec), and eluted after removal of the column from the magnetic field. The CD8+ T cells are collected, and, optionally, stored, until used in a method described herein for the production of genetically modified CD8+ T cells.

In other embodiments, methods of producing a CD8+ cytotoxic T lymphocyte (CTL) may include (a) isolating CD8+ T cells from peripheral blood mononuclear cells (PBMC), (b) activating the isolated CD8+ T cells with an anti-CD3 antibody and an anti-CD28 antibody, (c) introducing a nucleic acid into the activated CD8+ T cells, (d) expanding the transformed CD8+ T cells, and (e) harvesting the transformed CD8+ T cells, wherein step (a) through the step (e) are performed within 6 days. In another aspect, the method takes no longer than 6 days to complete. In an aspect, the method may take 1, 2, 3, 4, 5, 6, 7, 10 or 14 days to complete. The method may further comprise cryopreserving the harvested T-cells. In another aspect, the total time to complete steps (b), (c), (d) and (e) may be from about 6 days to about to about 10 days. In another aspect, activation (b) may be carried out within a period of from about 15 hours to about 24 hours, transduction (c) may be carried out from about 20 hours to about 28 hours, and expansion (d) may be carried out from about 5 days to about 6 days.

In an embodiment, the peripheral blood mononuclear cells (PBMC) may be obtained from a healthy donor. The peripheral blood mononuclear cells (PBMC) may be obtained from a patient. The peripheral blood mononuclear cells (PBMC) may be autologous or allogenic.

In an embodiment, the number of the isolated CD8+ T cells may be from about 1×10⁸ to about 3×10⁹, from about 2×10⁸ to about 3×10⁹, from about 3×10⁸ to about 3×10⁹, from about 4×10⁸ to about 3×10⁹, from about 5×10⁸ to about 3×10⁹, from about 6×10⁸ to about 3×10⁹, from about 7×10⁸ to about 3×10⁹, from about 8×10⁸ to about 3×10⁹, from about 9×10⁸ to about 3×10⁹, from about 1×10⁹ to about 3×10⁹, from about 1×10⁹ to about 2.5×10⁹, from about 1×10⁹ to about 2×10⁹, or from about 1×10⁹ to about 1.5×10⁹. The number of the isolated CD8+ T cells may be about 1×10⁸ cells, 2×10⁸ cells, 3×10⁸ cells, 4×10⁸ cells, 5×10⁸ cells, 6×10⁸ cells, 7×10⁸ cells, 8×10⁸ cells, 9×10⁸ cells, 1×10⁹ cells, 2×10⁹ cells, 3×10⁹ cells, 4×10⁹ cells, 5×10⁹ cells, 6×10⁹ cells, 7×10⁹ cells, 8×10⁹ cells, 9×10⁹ cells, or 1×10¹⁰ cells.

In an embodiment, the purity of the isolated CD8+ T cells in a preparation may be from about 60% to about 100%, from about 65% to about 100%, from about 70% to about 100%, from about 75% to about 100%, from about 80% to about 100%, from about 85% to about 100%, from about 90% to about 100%, from about 95% to about 100%, from about 96% to about 100%, from about 97% to about 100%, from about 98% to about 100%, or from about 99% to about 100%. The purity of the isolated CD8+ T cells in a preparation may be about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%.

In an embodiment, the CD8+ T cells are CD4+.

### T Cell Activation

The T cells may be activated, wherein the T cells that have been sufficiently stimulated to induce detectable cellular proliferation. Activation can also be associated with induced cytokine production, and detectable effector functions. Signals generated through the TCR alone are insufficient for full activation of the T cell and one or more secondary or costimulatory signals are also required. Thus, T cell activation comprises a primary stimulation signal through the TCR/CD3 complex and one or more secondary costimulatory signals. Co-stimulation can be evidenced by proliferation and/or cytokine production by T cells that have received a primary activation signal, such as stimulation through the CD3/TCR complex or through CD2.

A population of T cells may be induced to proliferate by activating T cells and stimulating an accessory molecule on the surface of T cells with a ligand, which binds the accessory molecule. Activation of a population of T cells may be accomplished by contacting T cells with a first agent which stimulates a TCR/CD3 complex-associated signal in the T cells. Stimulation of the TCR/CD3 complex-associated signal in a T cell may be accomplished either by ligation of the T cell receptor (TCR)/CD3 complex or the CD2 surface protein, or by directly stimulating receptor-coupled signalling pathways. Thus, an anti-CD3 antibody, an anti-CD2 antibody, or a protein kinase C activator in conjunction with a calcium ionophore may be used to activate a population of T cells. Both anti-CD3 and anti-CD2 antibodies are known in the art and are commercially available.

To induce proliferation, an activated population of T cells may be contacted with a second agent, which stimulates an accessory molecule on the surface of the T cells. For example, a population of CD4+ T cells can be stimulated to proliferate with an anti-CD28 antibody directed to the CD28 molecule on the surface of the T cells. Anti-CD28 antibodies are known in the art and are commercially available.

Alternatively, CD4+ T cells can be stimulated with a natural ligand for CD28, such as B7-1 and B7-2. The natural ligand can be soluble, on a cell membrane, or coupled to a solid phase surface. Proliferation of a population of CD8+ T cells may be accomplished by use of a monoclonal antibody ES5.2D8, which binds to CD9, an accessory molecule having a molecular weight of about 27 kD present on activated T cells. Alternatively, proliferation of an activated population of T cells can be induced by stimulation of one or more intracellular signals, which result from ligation of an accessory molecule, such as CD28.

The agent providing the primary activation signal and the agent providing the costimulatory agent can be added either in soluble form or coupled to a solid phase surface. In a preferred embodiment, the two agents may be coupled to the same solid phase surface.

Following activation and stimulation of an accessory molecule on the surface of the T cells, the progress of proliferation of the T cells in response to continuing exposure to the ligand or other agent, which acts intracellularly to simulate a pathway mediated by the accessory molecule, may be monitored. When the rate of T cell proliferation decreases, T cells may be reactivated and re-stimulated, such as with additional anti-CD3 antibody and a co-stimulatory ligand, to induce further proliferation. The rate of T cell proliferation may be monitored by examining cell size. Alternatively, T cell proliferation may be monitored by assaying for expression of cell surface molecules in response to exposure to the ligand or other agent, such as B7-1 or B7-2. The monitoring and re-stimulation of T cells can be repeated for sustained proliferation to produce a population of T cells increased in number from about 100- to about 100,000-fold over the original T cell population.

The anti-CD3 antibody and the anti-CD28 antibody each may have a concentration of no more than about 0.1 μg/ml, no more than about 0.2 μg/ml, no more than about 0.3 μg/ml, no more than about 0.4 μg/ml, no more than about 0.5 μg/ml, no more than about 0.6 μg/ml, no more than about 0.7 μg/ml, no more than about 0.8 μg/ml, no more than about 0.9 μg/ml, no more than about 1.0 μg/ml, no more than about 2.0 μg/ml, no more than about 4.0 μg/ml, no more than about 6.0 μg/ml, no more than about 8.0 μg/ml, or no more than about 10.0 μg/ml.

The anti-CD3 antibody and the anti-CD28 antibody each may have a concentration of from about 0.1 μg/ml to about 1.0 μg/ml, about 0.1 μg/ml to about 0.8 μg/ml, about 0.1 μg/ml to about 0.6 μg/ml, about 0.1 μg/ml to about 0.5 μg/ml, about 0.1 μg/ml to about 0.25 μg/ml, about 0.2 μg/ml to about 0.5 μg/ml, about 0.2 μg/ml to about 0.3 μg/ml, about 0.3 μg/ml to about 0.5 μg/ml, about 0.3 μg/ml to about 0.4 μg/ml, about 0.2 μg/ml to about 0.5 μg/ml, about 0.1 μg/ml to about 10.0 μg/ml, about 0.1 μg/ml to about 8.0 μg/ml, about 0.1 μg/ml to about 6.0 μg/ml, about 0.1 μg/ml to about 4.0 μg/ml, or about 0.1 μg/ml to about 2.0 μg/ml.

The anti-CD3 antibody and the anti-CD28 antibody may be immobilized on a solid phase support. The solid phase support may be in the form of a bead, box, column, cylinder, disc, dish (e.g., glass dish, PETRI dish), fibre, film, filter, microtiter plate (e.g., 96-well microtiter plate), multi-bladed stick, net, pellet, plate, ring, rod, roll, sheet, slide, stick, tray, tube, or vial. The solid phase support can be a singular discrete body (e.g., a single tube, a single bead), any number of a plurality of substrate bodies (e.g., a rack of 10 tubes, several beads), or combinations thereof (e.g., a tray comprises a plurality of microtiter plates, a column filled with beads, a microtiter plate filed with beads). Conti et al. (2003) Current Protocols in Cytometry John Wiley & Sons, Inc. The solid phase support may be a surface of a bead, tube, tank, tray, dish, a plate, a flask, or a bag. The solid phase support may be an array.

The activation of the T cells may be carried for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 hours. The activation of the T cells may be carried for about 1-10 hours, 11-30 hours, 15-25 hours, 31-50 hours, 51-100 hours, or 101-120 hours. The T-cells may be CD8+ T-cells.

The activation of the T cells may be conducted at a temperature between about 0° C. and about 42° C. The activation of the T cells may be conducted at a temperature at about 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., or 41° C. The activation of the T cells may be conducted at a temperature between about 30° C. and about 40° C. The T-cells may be CD8+ T-cells.

Conventional methods of activating T cells may involve an open-system and a labor-intensive process using either commercially available beads or non-tissue culture treated 24-well or 6-well plates coated with anti-CD3 and anti-CD28 antibodies (“plate-bound”) at a concentration of 1 ug/mL each. Open system methods, however, may take a relatively long time, e.g., about 8 hours, to complete. To simplify the open-system and the labor-intensive process, the inventors streamlined the system to a process adaptable to a closed-system that can be combined with containers, e.g., bags, of commercially available closed system, e.g., GRex® (cell expansion) system and Xuri® cell expansion system, resulting in comparable T cell activation profile, transducibility of T cells, and functionality of the end-product with that of T cells activated using the conventional methods. In addition, methods of the present disclosure, e.g., flask-bound method, may take a relatively short time, e.g., about 1 hour, to complete, which is about 8 times faster than the conventional methods.

The closed system may be CliniMACS Prodigy® (closed and automated platform for cell manufacturing), WAVE (XUIRI®) Bioreactor (cell expansion system), WAVE (XUIRI®) Bioreactor (cell expansion system) in combination with BioSafe Sepax® II (cell separation system), G-Rex® closed system (cell expansion system), or G-Rex® closed system (cell expansion system) in combination with BioSafe Sepax® II (cell separation system).

### T Cell Transformation

Nucleic acids encoding recombinant proteins, e.g., CARs, TCRs, cytokines, antibodies, and/or bi-specific binding molecules, may be introduced into the T cells as naked DNA or in a suitable vector, such as a viral vector. The T-cells may be CD8+ T-cells. Methods of stably transfecting T cells by electroporation or other non-viral gene transfer (such as, but not limited to, sonoporation) using naked DNA are known in the art. See, e.g., U.S. Pat. No. 6,410,319; T Cell Protocols (2^{nd} Edition) De Libero (Ed.) 2009 Humana Press; Molecular Cloning: A Laboratory Manual (4^{th} Edition) Green & Sambrook (Ed.) 2012 Cold Spring Harbor Press. Naked DNA generally refers to the DNA encoding recombinant proteins contained in a plasmid expression vector in proper orientation for expression. Advantageously, the use of naked DNA reduces the time required to produce T cells expressing the recombinant proteins.

A viral vector (e.g., a retroviral vector, adenoviral vector, adeno-associated viral vector, or lentiviral vector) can be used to introduce nucleic acids encoding recombinant proteins into the CD8+ T cells. Suitable vectors for use in accordance with the method of the present disclosure are non-replicating in the subject's T cells. A large number of vectors are known that are based on viruses, where the copy number of the virus maintained in the cell is low enough to maintain the viability of the cell. Illustrative vectors that may be used in the methods described herein include the pFB-neo vectors (STRATAGENE®) as well as vectors based on gamma-retrovirus, lentivirus (LV), e.g., human immunodeficiency virus (HIV), simian vacuolating virus 40 (SV40), Epstein-Barr virus (EBV), herpes simplex virus (HSV), or bovine papillomaviruses (BPV). Methods and materials for stably transfecting T cells with viral vectors are known in the art. Viral Vectors for Gene Therapy: Methods and Protocols Machida (Ed.) 2003 Humana Press. See, e.g., T Cell Protocols (2^{nd} Edition) De Libero (Ed.) 2009 Humana Press; Molecular Cloning: A Laboratory Manual (4^{th} Edition) Green & Sambrook (Ed.) 2012 Cold Spring Harbor Press.

### Lentiviral Viral Vectors

The lentiviral vectors used herein contain several elements previously shown to enhance vector function, including a central polypurine tract (cPPT) for improved replication and nuclear import, a promoter from the murine stem cell virus (MSCV), which has been shown to lessen vector silencing in some cell types, a woodchuck hepatitis virus posttranscriptional responsive element (WPRE) (SEQ ID NO: 174) for improved transcriptional termination, and the backbone was a deleted 3′-LTR self-inactivating (SIN) vector design that may have improved safety, sustained gene expression and anti-silencing properties (Yang et al. Gene Therapy (2008) 15, 1411-1423, the content of which is incorporated by reference in its entirety).

In an aspect, vectors, constructs, or sequences described herein comprise mutated forms of WPRE. In another aspect, sequences or vectors described herein comprise mutations in WPRE version 1, e.g., WPREmut1 (SEQ ID NO: 175), or WPRE version 2, e.g., WPREmut2 (SEQ ID NO: 176). In an aspect, WPRE mutants comprise at most one mutation, at most two mutations, at most three mutations, at least four mutations, or at most five mutations. In an aspect, vectors, constructs, or sequences described herein do not comprise WPRE.

In another aspect, vectors, constructs, or sequences described herein do not include an X protein promoter.

To obtain optimal co-expression levels of TCRαβ and CD8αβ in the transduced γδ T cells or T cells, lentiviral vectors with various designs may be generated. T cells may be transduced with two separate lentiviral vectors (2-in-1) expressing TCRαβ or CD8αβ and a single lentiviral vector (4-in-1) co-expressing TCRαβ and CD8αβ or and a single lentiviral vector (3-in-1) co-expressing TCRαβ and CD8α in the absence of CD8β. In the 4-in-1 or 3-in-1 vector, the nucleotides encoding TCRα chain, TCRβ chain, CD8α chain, and/or CD8β chain may be shuffled in various orders. Various 4-in-1 or 3-in-1 vectors, thus generated, may be used to transduce γδ T cells or αβ T cells, followed by measuring TCR/CD8 co-expression levels of the transduced cells using techniques known in the art, e.g., flow cytometry.

To generate lentiviral vectors co-expressing TCRαβ and CD8αβ, a nucleotide encoding furin-linker-2A peptide may be positioned between TCRα chain and TCRβ chain, between CD8α chain and CD8β chain, and between a TCR chain and a CD8 chain to enable highly efficient gene expression. The 2A peptide may be selected from P2A, T2A, E2A, or F2A.

Lentiviral viral vectors may also contain post-transcriptional regulatory element (PRE), such as Woodchuck PRE (WPRE) (SEQ ID NO: 174) to enhance the expression of the transgene by increasing both nuclear and cytoplasmic mRNA levels. One or more regulatory elements including mouse RNA transport element (RTE), the constitutive transport element (CTE) of the simian retrovirus type 1 (SRV-1), and the 5′ untranslated region of the human heat shock protein 70 (Hsp70 5′UTR) may also be used and/or in combination with WPRE to increase transgene expression.

Lentiviral vectors may be pseudotyped with RD114TR (SEQ ID NO: 177), which is a chimeric glycoprotein containing an extracellular and transmembrane domain of feline endogenous virus (RD114) fused to cytoplasmic tail (TR) of murine leukemia virus. Other viral envelop proteins, such as VSV-G env, MLV 4070A env, RD114 env, chimeric envelope protein RD114pro, baculovirus GP64 env, or GALV env, or derivatives thereof, may also be used.

Once it is established that the transfected or transduced T cell is capable of expressing the recombinant proteins, e.g., CARs and TCRs, as surface membrane proteins with the desired regulation and at a desired level, it can be determined whether the CARs and TCRs are functional in the host cell to provide for the desired signal induction. Subsequently, the transduced T cells may be reintroduced or administered to the subject to activate anti-tumor responses in the subject.

TABLE 1

| SEQ ID NO: | Description | Sequence |
|---|---|---|
| 163 | CD8α1 | MALPVTALLLPLALLLHAARPSQFRVSPLDRTWNLGET |
| | | VELKCQVLLSNPTSGCSWLFQPRGAAASPTFLLYLSQN |
| | | KPKAAEGLDTQRFSGKRLGDTFVLTLSDFRRENEGYYF |
| | | CSALSNSIMYFSHFVPVFLPAKPTTTPAPRPPTPAPTIASQ |
| | | PLSLRPEACRPAAGGAVHTRGLDFACDIYIWAPLAGTC |
| | | GVLLLSLVITLYCNHRNRRRVCKCPRPVVKSGDKPSLS |
| | | ARYV |
| 164 | CD8α2 | MALPVTALLLPLALLLHAARPSQFRVSPLDRTWNLGET |
| | | VELKCQVLLSNPTSGCSWLFQPRGAAASPTFLLYLSQN |
| | | KPKAAEGLDTQRFSGKRLGDTFVLTLSDFRRENEGCYF |
| | | CSALSNSIMYFSHFVPVFLPAKPTTTPAPRPPTPAPTIASQ |
| | | PLSLRPEACRPAAGGAVHTRGLDFACDIYIWAPLAGTC |
| | | GVLLLSLVITLYCNHRNRRRVCKCPRPVVKSGDKPSLS |
| | | ARYV |
| 165 | m1CD8α | MALPVTALLLPLALLLHAARPSQFRVSPLDRTWNLGET |
| | | VELKCQVLLSNPTSGCSWLFQPRGAAASPTFLLYLSQN |
| | | KPKAAEGLDTQRFSGKRLGDTFVLTLSDFRRENEGYYF |
| | | CSALSNSIMYFSHFVPVFLPASVVDFLPTTAQPTKKSTL |
| | | KKRVCRLPRPETQKGPLCSPIYIWAPLAGTCGVLLLSLVI |
| | | TLYCNHRNRRRVCKCPRPVVKSGDKPSLSARYV |
| 166 | m2CD8α | MALPVTALLLPLALLLHAARPSQFRVSPLDRTWNLGET |
| | | VELKCQVLLSNPTSGCSWLFQPRGAAASPTFLLYLSQN |
| | | KPKAAEGLDTQRFSGKRLGDTFVLTLSDFRRENEGCYF |
| | | CSALSNSIMYFSHFVPVFLPASVVDFLPTTAQPTKKSTL |
| | | KKRVCRLPRPETQKGPLCSPIYIWAPLAGTCGVLLLSLVI |
| | | TLYCNHRNRRRVCKCPRPVVKSGDKPSLSARYV |
| 167 | CD8β1 | MRPRLWLLLAAQLTVLHGNSVLQQTPAYIKVQTNKMV |
| | | MLSCEAKISLSNMRIYWLRQRQAPSSDSHHEFLALWDS |
| | | AKGTIHGEEVEQEKIAVFRDASRFILNLTSVKPEDSGIYF |
| | | CMIVGSPELTFGKGTQLSVVDFLPTTAQPTKKSTLKKRV |
| | | CRLPRPETQKGPLCSPITLGLLVAGVLVLLVSLGVAIHL |
| | | CCRRRRARLRFMKQPQGEGISGTFVPQCLHGYYSNTTT |
| | | SQKLLNPWILKT |
| 168 | CD8β2 | MRPRLWLLLAAQLTVLHGNSVLQQTPAYIKVQTNKMV |
| | | MLSCEAKISLSNMRIYWLRQRQAPSSDSHHEFLALWDS |
| | | AKGTIHGEEVEQEKIAVFRDASRFILNLTSVKPEDSGIYF |
| | | CMIVGSPELTFGKGTQLSVVDFLPTTAQPTKKSTLKKRV |
| | | CRLPRPETQKGLKGKVYQEPLSPNACMDTTAILQPHRS |
| | | CLTHGS |
| 169 | CD8β3 | LQQTPAYIKVQTNKMVMLSCEAKISLSNMRIYWLRQRQ |
| | | APSSDSHHEFLALWDSAKGTIHGEEVEQEKIAVFRDASR |
| | | FILNLTSVKPEDSGIYFCMIVGSPELTFGKGTQLSVVDFL |
| | | PTTAQPTKKSTLKKRVCRLPRPETQKGPLCSPITLGLLV |
| | | AGVLVLLVSLGVAIHLCCRRRRARLRFMKQFYK |
| 170 | CD8β4 | LQQTPAYIKVQTNKMVMLSCEAKISLSNMRIYWLRQRQ |
| | | APSSDSHHEFLALWDSAKGTIHGEEVEQEKIAVFRDASR |
| | | FILNLTSVKPEDSGIYFCMIVGSPELTFGKGTQLSVVDFL |
| | | PTTAQPTKKSTLKKRVCRLPRPETQKGPLCSPITLGLLV |
| | | AGVLVLLVSLGVAIHLCCRRRRARLRFMKQLRLHPLEK |
| | | CSRMDY |
| 171 | CD8β5 | LQQTPAYIKVQTNKMVMLSCEAKISLSNMRIYWLRQRQ |
| | | APSSDSHHEFLALWDSAKGTIHGEEVEQEKIAVFRDASR |
| | | FILNLTSVKPEDSGIYFCMIVGSPELTFGKGTQLSVVDFL |
| | | PTTAQPTKKSTLKKRVCRLPRPETQKGPLCSPITLGLLV |
| | | AGVLVLLVSLGVAIHLCCRRRRARLRFMKQKFNIVCLK |
| | | ISGFTTCCCFQILQISREYGFGVLLQKDIGQ |
| 172 | CD8β6 | LQQTPAYIKVQTNKMVMLSCEAKISLSNMRIYWLRQRQ |
| | | APSSDSHHEFLALWDSAKGTIHGEEVEQEKIAVFRDASR |
| | | FILNLTSVKPEDSGIYFCMIVGSPELTFGKGTQLSVVDFL |
| | | PTTAQPTKKSTLKKRVCRLPRPETQKGPLCSPITLGLLV |
| | | AGVLVLLVSLGVAIHLCCRRRRARLRFMKQKFNIVCLK |
| | | ISGFTTCCCFQILQISREYGFGVLLQKDIGQ |
| 173 | CD8β7 | LQQTPAYIKVQTNKMVMLSCEAKISLSNMRIYWLRQRQ |
| | | APSSDSHHEFLALWDSAKGTIHGEEVEQEKIAVFRDASR |
| | | FILNLTSVKPEDSGIYFCMIVGSPELTFGKGTQLSVVDFL |
| | | PTTAQPTKKSTLKKRVCRLPRPETQKGPLCSPITLGLLV |
| | | AGVLVLLVSLGVAIHLCCRRRRARLRFMKQPQGEGISG |
| | | TFVPQCLHGYYSNTTTSQKLLNPWILKT |
| 174 | WPRE | cagtctgacgtacgcgtaatcaacctctggattacaaaatttgtgaaagattga |
| | | ctggtattcttaactatgttgctccttttacgctatgtggatacgctgctttaatgcct |
| | | ttgtatcatgctattgcttcccgtatggctttcattttctcctccttgtataaatcctggt |
| | | tgctgtctctttatgaggagttgtggcccgttgtcaggcaacgtggcgtggtgtg |
| | | cactgtgtttgctgacgcaacccccactggttggggcattgccaccacctgtca |
| | | gctcctttccgggactttcgctttccccctccctattgccacggcggaactcatc |
| | | gccgcctgccttgcccgctgctggacaggggctcggctgttgggcactgaca |
| | | attccgtggtgttgtcggggaagctgacgtcctttccatggctgctcgcctgtgtt |
| | | gccacctggattctgcgcgggacgtccttctgctacgtcccttcggccctcaat |
| | | ccagcggaccttccttcccgcggcctgctgccggctctgcggcctcttccgcgt |
| | | cttcgccttcgccctcagacgagtcggatctccctttgggccgcctccccgcc |
| 175 | WPREmut1 | cagtctgacgtacgcgtaatcaacctctggattacaaaatttgtgaaagattga |
| | | ctggtattcttaactatgttgctccttttacgctatgtggatacgctgctttaatgcct |
| | | ttgtatcatgctattgcttcccgtatggctttcattttctcctccttgtataaatcctggt |
| | | tgctgtctctttatgaggagttgtggcccgttgtcaggcaacgtggcgtggtgtg |
| | | cactgtgtttgctgacgcaacccccactggttggggcattgccaccacctgtca |
| | | gctcctttccgggactttcgctttccccctccctattgccacggcggaactcatc |
| | | gccgcctgccttgcccgctgctggacaggggctcggctgttgggcactgaca |
| | | attccgtggtgttgtcggggaaatcatcgtcctttccttggctgctcgcctgtgttg |
| | | ccacctggattctgcgcgggacgtccttctgctacgtcccttcggccctcaatc |
| | | cagcggaccttccttcccgcggcctgctgccggctctgcggcctcttccgcgtc |
| | | ttcgccttcgccctcagacgagtcggatctccctttgggccgcctccccgcc |
| 176 | WPREmut2 | Gagcatcttaccgccatttatacccatatttgttctgtttttcttgatttgggtataca |
| | | tttaaatgttaataaaacaaaatggtggggcaatcatttacattttttgggatatg |
| | | taattactagttcaggtgtattgccacaagacaaacttgttaagaaactttcccg |
| | | ttatttacgctctgttcctgttaatcaacctctggattacaaaatttgtgaaagattg |
| | | actgatattcttaactttgttgctccttttacgctgtgtggatttgctgctttattgcctc |
| | | tgtatcttgctattgcttcccgtacggctttcgttttctcctccttgtataaatcctggtt |
| | | gctgtctctttttgaggagttgtggcccgttgtccgtcaacgtggcgtggtgtgct |
| | | ctgtgtttgctgacgcaacccccactggctggggcattgccaccacctgtcaa |
| | | ctcctttctgggactttcgctttccccctcccgatcgccacggcagaactcatcg |
| | | ccgcctgccttgcccgctgctggacaggggctaggttgctgggcactgataat |
| | | tccgtggtgttgtc |
| 177 | RD114TR | MKLPTGMVILCSLIIVRAGFDDPRKAIALVQKQHGKPCE |
| | | CSGGQVSEAPPNSIQQVTCPGKTAYLMTNQKWKCRV |
| | | TPKISPSGGELQNCPCNTFQDSMHSSCYTEYRQCRRI |
| | | NKTYYTATLLKIRSGSLNEVQILQNPNQLLQSPCRGSIN |
| | | QPVCWSATAPIHISDGGGPLDTKRVWTVQKRLEQIHK |
| | | AMTPELQYHPLALPKVRDDLSLDARTFDILNTTFRLLQ |
| | | MSNFSLAQDCWLCLKLGTPTPLAIPTPSLTYSLADSLA |
| | | NASCQIIPPLLVQPMQFSNSSCLSSPFINDTEQIDLGAV |
| | | TFTNCTSVANVSSPLCALNGSVFLCGNNMAYTYLPQN |
| | | WTRLCVQASLLPDIDINPGDEPVPIPAIDHYIHRPKRAV |
| | | QFIPLLAGLGITAAFTTGATGLGVSVTQYTKLSHQLISD |
| | | VQVLSGTIQDLQDQVDSLAEVVLQNRRGLDLLTAEQG |
| | | GICLALQEKCCFYANKSGIVRNKIRTLQEELQKRRESLA |
| | | SNPLWTGLQGFLPYLLPLLGPLLTLLLILTIGPCVFNRLV |
| | | QFVKDRISVVQALVLTQQYHQLKPL |

### Engineered CD4+CD8+ T Cells

CD8 is a membrane-anchored glycoprotein that functions as a coreceptor for antigen recognition of the peptide/MHC class I complexes by T cell receptors (TCR) and plays an important role in T cell development in the thymus and T cell activation in the periphery. Functional CD8 is a dimeric protein made of either two a chains (CD8αα) or an α chain and a β chain (CD8αβ), and the surface expression of the β chain may require its association with the co-expressed a chain to form the CD8αβ heterodimer. CD8αα and CD8αβ may be differentially expressed on a variety of lymphocytes. CD8αβ is expressed predominantly on the surface of αβTCR+ T cells and thymocytes, and CD8αα on a subset of αβTCR+, γδTCR+ intestinal intraepithelial lymphocytes, NK cells, dendritic cells, and a small fraction of CD4+ T cells.

In an embodiment, the T cells may be a γδ T cell or an αβ T cell that express exogenous CD8αβ heterodimer or exogenous CD8α homodimer, or variants thereof, for example, as shown in Table 1, CD8α polypeptide may be CD8α1 (SEQ ID NO: 163), CD8α2 (SEQ ID NO: 164), m1CD8α (SEQ ID NO: 165), or m2CD8α (SEQ ID NO: 166), and CD8β polypeptide may be CD8β1 (SEQ ID NO: 167), CD8β2 (SEQ ID NO: 168), CD8β3 (SEQ ID NO: 169), CD8β4 (SEQ ID NO: 170), CD8β5 (SEQ ID NO: 171), CD8β6 (SEQ ID NO: 172), or CD8β7 (SEQ ID NO: 173).

CD8α sequences may generally have a sufficient portion of the immunoglobulin domain to be able to bind to MHC. Generally, CD8α molecules may contain all or a substantial part of immunoglobulin domain of CD8α, e.g., CD8α1 (SEQ ID NO: 163), but in an aspect may contain at least 10, 15, 20, 25, 30, 35, 40, 45, 50, 55, 60, 65, 70, 75, 80, 85, 90, 95, 100, 105, 110 or 115 amino acids of the immunoglobulin domain. The CD8α molecules of the present disclosure may be preferably dimers (e.g., CD8αα or CD8αβ), although CD8α monomer may be included within the scope of the present disclosure. In an aspect, CD8α of the present disclosure may comprise CD8α1 (SEQ ID NO: 163) and CD8α2 (SEQ ID NO: 164).

CD8α and β subunits may have similar structural motifs, including an Ig-like domain, a stalk region of 30-40 amino acids, a transmembrane region, and a short cytoplasmic domain of about 20 amino acids. CD8α and β chains have two and one N-linked glycosylation sites, respectively, in the Ig-like domains where they share<20% identity in their amino acid sequences. The CD8β stalk region is 10-13 amino acids shorter than the CD8α stalk and is highly glycosylated with O-linked carbohydrates. These carbohydrates on the β, but not the α, stalk region appear to be quite heterogeneous due to complex sialylations, which may be differentially regulated during the developmental stages of thymocytes and upon activation of T cells. Glycan adducts have been shown to play regulatory roles in the functions of glycoproteins and in immune responses. Glycans proximal to transmembrane domains can affect the orientation of adjacent motifs. The unique biochemical properties of the CD8β chain stalk region may present a plausible candidate for modulating the coreceptor function.

Engineered T-cells may express exogenous CD8 polypeptides described herein. For example, a T-cell may co-express a T-cell Receptor (TCR) and exogenous CD8 polypeptides described herein. T-cells may also express a chimeric antigen receptor (CAR), CAR-analogues, or CAR derivatives.

The T-cell may be a αβ T cell, γδ T cell, natural killer T cell, or a combination thereof if in a population. The T cell may be a CD4+ T cell, CD8+ T cell, or a CD4+/CD8+ T cell.

### Engineered T Cells Expressing Exogenous T-Cell Receptors (TCR)

T-cell may co-express a T-cell receptor (TCR), antigen binding protein, or both, with exogenous CD8 polypeptides described herein, including, but are not limited to, those listed in Table 1 (SEQ ID NOs: 163-173). Further, a T-cell may express a TCRs and antigen binding proteins described in U.S. Patent Application Publication No. 2017/0267738; U.S. Patent Application Publication No. 2017/0312350; U.S. Patent Application Publication No. 2018/0051080; U.S. Patent Application Publication No. 2018/0164315; U.S. Patent Application Publication No. 2018/0161396; U.S. Patent Application Publication No. 2018/0162922; U.S. Patent Application Publication No. 2018/0273602; U.S. Patent Application Publication No. 2019/0016801; U.S. Patent Application Publication No. 2019/0002556; U.S. Patent Application Publication No. 2019/0135914; U.S. Pat. Nos. 10,538,573; 10,626,160; U.S. Patent Application Publication No. 2019/0321478; U.S. Patent Application Publication No. 2019/0256572; U.S. Pat. Nos. 10,550,182; 10,526,407; U.S. Patent Application Publication No. 2019/0284276; U.S. Patent Application Publication No. 2019/0016802; U.S. Patent Application Publication No. 2019/0016803; U.S. Patent Application Publication No. 2019/0016804; U.S. Pat. No. 10,583,573; U.S. Patent Application Publication No. 2020/0339652; U.S. Pat. Nos. 10,537,624; 10,596,242; U.S. Patent Application Publication No. 2020/0188497; U.S. Pat. No. 10,800,845; U.S. Patent Application Publication No. 2020/0385468; U.S. Pat. Nos. 10,527,623; 10,725,044; U.S. Patent Application Publication No. 2020/0249233; U.S. Pat. No. 10,702,609; U.S. Patent Application Publication No. 2020/0254106; U.S. Pat. No. 10,800,832; U.S. Patent Application Publication No. 2020/0123221; U.S. Pat. Nos. 10,590,194; 10,723,796; U.S. Patent Application Publication No. 2020/0140540; U.S. Pat. No. 10,618,956; U.S. Patent Application Publication No. 2020/0207849; U.S. Patent Application Publication No. 2020/0088726; and U.S. Patent Application Publication No. 2020/0384028; the contents of each of these publications and sequence listings described therein are herein incorporated by reference in their entireties. The T-cell may be a αβ T cell, γδ T cell, natural killer T cell. Natural killer cell. In an embodiment, TCRs described herein are single-chain TCRs or soluble TCRs.

### T Cell Expansion

Following T cell transduction, the cells may be propagated for days, weeks, or months ex vivo as a bulk population within about 1, 2, 3, 4, 5 days or more following gene transfer into cells. In a further aspect, following transduction, the transduced cells are cloned and a clone demonstrating presence of a single integrated or episomally maintained expression cassette or plasmid, and expression of recombinant proteins, e.g., TCRs, may be expanded ex vivo. The clones selected for expansion demonstrates the capacity to specifically recognize and lyse peptide-expressing target cells. The genetically modified T cells may be expanded by stimulation with IL-2, or other cytokines that bind the common gamma-chain (e.g., IFN-α, IL-4, IL-7, IL-9, IL-12, IL-15, IL-21, and others). The genetically modified T cells may be expanded by stimulation with artificial antigen presenting cells. The genetically modified T cells may be expanded on artificial antigen presenting cell or with an antibody, such as OKT3, which cross links CD3 on the T cell surface. Subsets of the genetically modified T cells may be deleted on artificial antigen presenting cell or with an antibody, such as Campath, which binds CD52 on the T cell surface. The genetically modified T cells may be cryopreserved.

Expansion of the T cells may be carried out in the presence of the T cell activation stimulus.

The expansion of the T cells may be carried out within a period of no more than about 1 day, no more than about 2 days, no more than about 3 days, no more than about 4 days, no more than about 5 days, or no more than about 6 days. The expansion of the T cells may be for about 1, 2, 3, 4, 5, or 6 days.

Expansion of the T cells may be carried out within a period of from about 1 day to about 6 days, about 1 day to about 5 days, about 1 day to about 4 days, about 1 day to about 3 days, about 1 day to about 2 days, or about 1 day.

Expansion of the T cells may be carried out in the presence of interferon (IFN)-α, interleukin (IL)-2, IL-4, IL-7, IL-9, IL-12, IL-15, IL-21, or a combination thereof. In an aspect, the expansion takes place in the presence of a combination IL-7 and IL-15.

Following expansion of the T cell population to sufficient numbers, the expanded T cells may be restored to the individual. The method of the present disclosure may also provide a renewable source of T cells. Thus, T cells from an individual can be expanded ex vivo, a portion of the expanded population can be re-administered to the individual and another portion can be frozen in aliquots for long term preservation, and subsequent expansion and administration to the individual. Similarly, a population of tumor-infiltrating lymphocytes can be obtained from an individual afflicted with cancer and the T cells stimulated to proliferate to sufficient numbers and restored to the individual.

The present disclosure may also pertain to compositions containing an agent that provides a costimulatory signal to a T cell for T cell expansion (e.g., an anti-CD28 antibody, B7-1 or B7-2 ligand), coupled to a solid phase surface, which may additionally include an agent that provides a primary activation signal to the T cell (e.g., an anti-CD3 antibody) coupled to the same solid phase surface. These agents may be preferably attached to beads or flasks or bags. Compositions comprising each agent coupled to different solid phase surfaces (e.g., an agent that provides a primary T cell activation signal coupled to a first solid phase surface and an agent that provides a costimulatory signal coupled to a second solid phase surface) may also be within the scope of this disclosure.

### Serum Free Medium in T Cell Manufacturing

As referred to herein, the term “serum-free media” or “serum-free culture medium” means that the growth media used is not supplemented with serum (e.g., human serum or bovine serum). In other words, no serum is added to the culture medium as an individually separate and distinct ingredient for the purpose of supporting the viability, activation, and growth of the cultured cells. Any suitable culture medium T cell growth media may be used for culturing the cells in accordance with the methods described herein. For example, a T cell growth media may include, but is not limited to, a sterile, low glucose solution that includes a suitable amount of buffer, magnesium, calcium, sodium pyruvate, and sodium bicarbonate. In one embodiment, the T cell growth media may include serum free media, e.g., OPTI-MEM®, D-MEM/F-12, 4CellNutri (Sartorius), AIM V (ThermoFisher), Physiologix (Nucleus Biologics), and/or viral production (VP) media (Life Technologies), but one skilled in the art would understand how to generate similar media. In contrast to typical methods for producing engineered T cells, the methods described herein use culture medium that may be not supplemented with serum (e.g., human or bovine).

VSV-G pseudotyped HIV and FIV vectors produced in human cells may be inactivated by human serum complement (DePolo et al. “VSV-G Pseudotyped Lentiviral Vector Particles Produced in Human Cells Are Inactivated by Human Serum,” Molecular Therapy (2000) 2:218-222; the content of which is hereby incorporated by reference in its entirety). In addition, reducing serum concentrations in culture media may result in a more sustainable process with equivalent growth kinetics and product quality (Tyagarajan et al. “Optimizing CAR-T Cell Manufacturing Processes during Pivotal Clinical Trials,” Molecular Therapy: Methods & Clinical Development, (2020) 16:136-144; the content of which is hereby incorporated by reference in its entirety). Therefore, it may be advantageous to include serum free media in T cell manufacturing process.

In an aspect, T cell activation, T cell transformation, and/or T cell expansion may be performed in serum free medium.

In an aspect, T cell activation may be performed in serum free medium or in the presence of serum.

In an aspect, T cell activation may be performed in serum free medium.

In an aspect, T cell transformation may be performed in serum free medium or in the presence of serum.

In an aspect, T cell transformation may be performed in serum free medium.

In one embodiment, T cell transformation performed in the absence of serum, e.g., in serum free medium, may increase frequency of CD8+ T cells, e.g., CD8+CD3+ T cells, in T cell products by from about 5% to about 60%, from about 5% to about 50%, from about 5% to about 45%, from about 5% to about 40%, from about 5% to about 35%, from about 5% to about 30%, from about 5% to about 25%, from about 5% to about 20%, from about 5% to about 15%, from about 5% to about 10%, from about 10% to about 50%, from about 10% to about 40%, from about 10% to about 30%, from about 10% to about 20%, from about 10% to about 15%, about 5%, about 10%, about 15%, about 20%, about 25%, about 30%, about 35%, about 40%, about 45%, about 50%, about 55%, or about 60%, as compared with that performed in the presence of serum.

In one embodiment, T cell transformation performed in the absence of serum, e.g., in serum free medium, may increase transduction efficiency of exogenous TCR in CD8, e.g., peptide/MHC Dextramer (Dex)+CD8+ T cells, by from about 5% to about 60%, from about 5% to about 50%, from about 5% to about 45%, from about 5% to about 40%, from about 5% to about 35%, from about 5% to about 30%, from about 5% to about 25%, from about 5% to about 20%, from about 5% to about 15%, from about 5% to about 10%, from about 10% to about 50%, from about 10% to about 40%, from about 10% to about 30%, from about 10% to about 20%, from about 10% to about 15%, about 5%, about 10%, about 15%, about 20%, about 25%, about 30%, about 35%, about 40%, about 45%, about 50%, about 55%, or about 60%, as compared with that performed in the presence of serum.

In an aspect, T cell expansion may be performed in serum free medium or in the presence of serum.

In an aspect, T cell expansion may be performed in serum free medium.

In an aspect, cryopreserved T cells may be thawed and rested in the presence of serum for about 2-8 hours, 2-6, hours, or 2-4 hours.

In an aspect, cryopreserved T cells may be thawed and rested in the presence of serum for about 2-4 hours, activated in the presence of serum, transduced in the absence of serum, and expanded in the presence of serum.

In an aspect, cryopreserved T cells may be thawed and rested in the presence of serum for about 2-4 hours, activated in the absence of serum, transduced in the absence of serum, and expanded in the presence of serum.

### T Cell Manufacturing in Closed Systems

Conventional methods of activating T cells may include an open-system and a labor-intensive process using either commercially available beads or non-tissue culture treated 24-well or 6-well plates coated with anti-CD3 and anti-CD28 antibodies (“plate-bound”) at a concentration of 1 ug/mL each. Open system methods, however, may take a relatively long time, e.g., about 8 hours, to complete. To simplify the open-system and the labor-intensive process, embodiments of the present disclosure may include a straightforward process adaptable to a closed-system that can be combined with containers, e.g., bags, of commercially available closed system, e.g., G-Rex™ system and Xuri™ cell expansion system, resulting in comparable T cell activation profile, transducibility of T cells, and functionality of the end-product with that of T cells activated using the conventional methods. In addition, methods of the present disclosure, e.g., flask-bound method, may take a relatively short time, e.g., about 1 hour, to complete, which is about 8 times faster than the conventional methods.

In some embodiments, T cell manufacturing process of the present disclosure may be carried out in any cell culture closed systems including commercially available systems, e.g., CliniMACS Prodigy™ (Miltenyi), WAVE (XURI™) Bioreactor (GE Biosciences) alone or in combination with BioSafe Sepax™ II, and G-Rex/GatheRex™ closed system (Wilson Wolf) alone or in combination with BioSafe Sepax™ II. G-Rex™-closed system is the expansion vessel and GatheRex™ is the pump for concentrating and harvesting.

CliniMACS Prodigy™ (Miltenyi)

CliniMACS Prodigy™ with TCT process software and the TS520 tubing set may allow closed-system processing for cell enrichment, transduction, washing and expansion. For example, MACS-CD4 and CD8-MicroBeads may be used for enrichment, TransACT beads, e.g., CD3/CD28 reagents, may be used for activation, lentiviral vectors expressing a recombinant TCR may be used for transduction, TexMACS medium-3%-HS-IL2 for culture and phosphate-buffered saline/ethylenediaminetetraacetic acid buffer for washing. This system may yield about 4-5×109 cells, contain automated protocols for manufacturing with chamber maximum ˜300 mL fill volume, and perform selection and activation (TransACT beads), transduction, and expansion over a 10 to 14-day process.

WAVE (Xuri™) Bioreactor (GE Biosciences)

WAVE (Xuri™) Bioreactor allows T cells to be cultured in culture bags, e.g., Xuri Cellbags, with and/or without perfusion. Medium bag for feeding may be 5-liter Hyclone Labtainer. Waste bag may be Mbag (purchased from GE Healthcare). This system may yield about 15-30×109 cells, use unicorn software that allows for culture control and monitoring, contain rocking tray that may hold from about 0.3-liter to about 25 liters, and perform perfusion function to maintain culture volume while mediating gas exchange and introducing fresh media and cytokines to cell culture.

WAVE (Xuri™) Bioreactor may include Xuri Bags for expansion, Saint Gobain's VueLife bags for thawing and resting, and VueLife AC bags for activation. WAVE (Xuri™) Bioreactor may be used in combination with other technologies, e.g., Sepax™ cell separation system (GE Biosciences) for culture washing and volume reduction steps. Sterile welder (Terumo BCT™) may be used for connecting sterile bags for solution transfer and heat sealer for sealing tubing.

Sepax™ cell separation system relies on a separation chamber that provides both separation through rotation of the syringe chamber (centrifugation) and component transfer through displacement of the syringe piston. An optical sensor measures the light absorbency of the separated components and manages the flow direction of each of them in the correct output container, for example, plasma, buffy coat, and red blood cells may be thus separated and collected from blood samples.

### Pharmaceutical Compositions

To facilitate administration, the transformed T cells according to the disclosure can be made into a pharmaceutical composition or made into an implant appropriate for administration in vivo, with pharmaceutically acceptable carriers or diluents. The means of making such a composition or an implant are described in the art. See, e.g., Remington's Pharmaceutical Sciences, 16th Ed., Mack, ed. (1980). The T-cells may be CD8+ T-cells.

The transduced T cells can be formulated into a preparation in semisolid or liquid form, such as a capsule, solution, infusion, or injection. Means known in the art can be utilized to prevent or minimize release and absorption of the composition until it reaches the target tissue or organ, or to ensure timed-release of the composition. Desirably, however, a pharmaceutically acceptable form is employed that does not hinder the cells from expressing the CARs or TCRs. Thus, desirably the transduced T cells can be made into a pharmaceutical composition comprising a carrier. The T cells produced by the methods described herein can be formulated with a physiologically acceptable carrier or excipient to prepare a pharmaceutical composition. The carrier and composition can be sterile. Preferred carriers include, for example, a balanced salt solution, preferably Hanks' balanced salt solution, or normal saline. The formulation should suit the mode of administration. Suitable pharmaceutically acceptable carriers include but are not limited to water, salt solutions (e.g., NaCl), saline, buffered saline, as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, which do not deleteriously react with the T-cells.

A composition of the present invention can be provided in unit dosage form wherein each dosage unit, e.g., an injection, contains a predetermined amount of the composition, alone or in appropriate combination with other active agents.

Compositions may comprise an effective amount of the isolated transduced T cells and be introduced into the subject such that long-term, specific, anti-tumor responses is achieved to reduce the size of a tumor or eliminate tumor growth or regrowth than would otherwise result in the absence of such treatment. For example, the amount of transduced T cells reintroduced into the subject causes an about 10%, about 20%, about 30%, about 40%, about 50%, about 60%, about 70%, about 80%, about 90%, about 95%, about 98%, or about 99% decrease in tumor size when compared to otherwise same conditions where the transduced T cells are not present.

Accordingly, the amount of transduced T cells administered may take into account the route of administration and should be such that a sufficient number of the transduced T cells will be introduced so as to achieve the desired therapeutic response. Furthermore, the amounts of each active agent included in the compositions described herein (e.g., the amount per each cell to be contacted or the amount per certain body weight) can vary in different applications. In general, the concentration of transduced T cells desirably should be sufficient to provide in the subject being treated, for example, effective amounts of transduced T cells may be about 1×10⁶ to about 1×10⁹ transduced T cells/m² (or kg) of a patient, even more desirably, from about 1×10⁷ to about 5×10⁸ transduced T cells/m² (or kg) of a patient. Any suitable amount can be utilized, e.g., greater than 5×10⁸ cells/m² (or kg) of a patient, or below, e.g., less than 1×10⁷ cells/m² (or kg) of a patient, as is necessary to achieve a therapeutic effect. The dosing schedule can be based on well-established cell-based therapies (See, e.g., U.S. Pat. No. 4,690,915), or an alternate continuous infusion strategy can be employed.

The T-cell products described herein may also be cryopreserved. Accordingly, cryopreserved T-cell compositions may comprise the genetically modified T-cells and a freezing media.

### Immunotherapy

Methods of treating a patient or individual having a cancer or in need of a treatment thereof, may comprise administering to the patient an effective amount of the expanded genetically modified T cells described herein. The patient or individual in need thereof may be a cancer patient. The cancer to be treated by the T cells descried herein may be hepatocellular carcinoma (HCC), colorectal carcinoma (CRC), glioblastoma (GB), gastric cancer (GC), esophageal cancer, non-small cell lung cancer (NSCLC), pancreatic cancer (PC), renal cell carcinoma (RCC), benign prostate hyperplasia (BPH), prostate cancer (PCA), ovarian cancer (OC), melanoma, breast cancer, chronic lymphocytic leukemia (CLL), Merkel cell carcinoma (MCC), small cell lung cancer (SCLC), Non-Hodgkin lymphoma (NHL), acute myeloid leukemia (AML), gallbladder cancer and cholangiocarcinoma (GBC, CCC), urinary bladder cancer (UBC), acute lymphocytic leukemia (ALL), uterine cancer (UEC), or a combination thereof.

T-cell based immunotherapy targets peptide epitopes derived from tumor-associated or tumor-specific proteins, which are presented by molecules of the major histocompatibility complex (MHC). The antigens that are recognized by the tumor specific T lymphocytes, that is, the epitopes thereof, can be molecules derived from all protein classes, such as enzymes, receptors, transcription factors, etc. which are expressed and as compared to unaltered cells of the same origin, usually up-regulated in cells of the respective tumor.

There are two classes of MHC-molecules, MHC class I and MHC class II. MHC class I molecules are composed of an alpha heavy chain and beta-2-microglobulin, MHC class II molecules of an alpha and a beta chain. Their three-dimensional conformation results in a binding groove, which is used for non-covalent interaction with peptides. MHC class I molecules can be found on most nucleated cells. They present peptides that result from proteolytic cleavage of predominantly endogenous proteins, defective ribosomal products (DRIPs) and larger peptides. However, peptides derived from endosomal compartments or exogenous sources are also frequently found on MHC class I molecules. This non-classical way of class I presentation is referred to as cross-presentation. MHC class II molecules can be found predominantly on professional antigen presenting cells (APCs), and primarily present peptides of exogenous or transmembrane proteins that are taken up by APCs, e.g., during endocytosis, and are subsequently processed.

Complexes of peptide and MHC class I are recognized by CD8+ T-cells bearing the appropriate T-cell receptor (TCR), whereas complexes of peptide and MHC class II molecules are recognized by CD4-positive-helper-T-cells bearing the appropriate TCR. It is well known that the TCR, the peptide and the MHC are thereby present in a stoichiometric amount of 1:1:1.

CD4+ helper T-cells play an important role in inducing and sustaining effective responses by CD8+ cytotoxic T-cells. The identification of CD4-positive T-cell epitopes derived from tumor associated antigens (TAA) is of great importance for the development of pharmaceutical products for triggering anti-tumor immune responses. At the tumor site, T helper cells, support a cytotoxic T-cell− (CTL−) friendly cytokine milieu and attract effector cells, e.g., CTLs, natural killer (NK) cells, macrophages, and granulocytes.

For an MHC class I peptide to trigger (elicit) a cellular immune response, it also must bind to an MHC-molecule. This process is dependent on the allele of the MHC-molecule and specific polymorphisms of the amino acid sequence of the peptide. MHC-class-1-binding peptides are usually 8-12 amino acid residues in length and usually contain two conserved residues (“anchors”) in their sequence that interact with the corresponding binding groove of the MHC-molecule. In this way, each MHC allele has a “binding motif” determining which peptides can bind specifically to the binding groove.

In the MHC class I dependent immune reaction, peptides not only have to be able to bind to certain MHC class I molecules expressed by tumor cells, they subsequently also have to be recognized by T-cells bearing specific T-cell receptors (TCR).

For proteins to be recognized by T-lymphocytes as tumor-specific or -associated antigens, and to be used in a therapy, particular prerequisites must be fulfilled. The antigen should be expressed mainly by tumor cells and not, or in comparably small amounts, by normal healthy tissues. The peptide should be over-presented by tumor cells as compared to normal healthy tissues. It is furthermore desirable that the respective antigen is not only present in a type of tumor, but also in high concentrations (e.g., copy numbers of the respective peptide per cell). Tumor-specific and tumor-associated antigens are often derived from proteins directly involved in transformation of a normal cell to a tumor cell due to their function, e.g., in cell cycle control or suppression of apoptosis. Additionally, downstream targets of the proteins directly causative for a transformation may be up-regulated and thus may be indirectly tumor-associated. Such indirect tumor-associated antigens may also be targets of a vaccination approach. Epitopes are present in the amino acid sequence of the antigen, in order to ensure that such a peptide (“immunogenic peptide”), being derived from a tumor associated antigen, and leads to an in vitro or in vivo T-cell-response.

TAAs are a starting point for the development of a T-cell based therapy including but not limited to tumor vaccines. The methods for identifying and characterizing the TAAs are usually based on the use of T-cells that can be isolated from patients or healthy subjects, or they are based on the generation of differential transcription profiles or differential peptide expression patterns between tumors and normal tissues. However, the identification of genes over-expressed in tumor tissues or human tumor cell lines, or selectively expressed in such tissues or cell lines, does not provide precise information as to the use of the antigens being transcribed from these genes in an immune therapy. This is because only an individual subpopulation of epitopes of these antigens are suitable for such an application since a T-cell with a corresponding TCR has to be present and the immunological tolerance for this particular epitope needs to be absent or minimal. In a very preferred embodiment of the description it is therefore important to select only those over- or selectively presented peptides against which a functional and/or a proliferating T-cell can be found. Such a functional T-cell is defined as a T-cell, which upon stimulation with a specific antigen can be clonally expanded and is able to execute effector functions (“effector T-cell”).

TAA peptides that are capable of use with the methods and embodiments described herein include, for example, those TAA peptides described in U.S. Patent Application Publication Nos. 2016/0187351; 2017/0165335; 2017/0035807; 2016/0280759; 2016/0287687; 2016/0346371; 2016/0368965; 2017/0022251; 2017/0002055; 2017/0029486; 2017/0037089; 2017/0136108; 2017/0101473; 2017/0096461; 2017/0165337; 2017/0189505; 2017/0173132; 2017/0296640; 2017/0253633; 2017/0260249; 2018/0051080, and 2018/0164315.

The T cells described herein selectively recognize cells which present a TAA peptide described in one of more of the patents and publications described above.

TAA that are capable of use with the methods and embodiments described herein include at least one amino acid sequence of SEQ ID NO: 1 to SEQ ID NO: 162. T cells selectively recognize cells which present a TAA peptide described in the amino acid sequences of SEQ ID NO: 1-162 or any of the patents or applications described herein.

TABLE 2
List of Tumor Associated Antigens (TAAs)

| SEQ ID | Amino Acid |
|---|---|
| NO: | Sequence |
| 1 | YLYDSETKNA |
| 2 | HLMDQPLSV |
| 3 | GLLKKINSV |
| 4 | FLVDGSSAL |
| 5 | FLFDGSANLV |
| 6 | FLYKIIDEL |
| 7 | FILDSAETTTL |
| 8 | SVDVSPPKV |
| 9 | VADKIHSV |
| 10 | IVDDLTINL |
| 11 | GLLEELVTV |
| 12 | TLDGAAVNQV |
| 13 | SVLEKEIYSI |
| 14 | LLDPKTIFL |
| 15 | YTFSGDVQL |
| 16 | YLMDDFSSL |
| 17 | KVWSDVTPL |
| 18 | LLWGHPRVALA |
| 19 | KIWEELSVLEV |
| 20 | LLIPFTIFM |
| 21 | FLIENLLAA |
| 22 | LLWGHPRVALA |
| 23 | FLLEREQLL |
| 24 | SLAETIFIV |
| 25 | TLLEGISRA |
| 26 | ILQDGQFLV |
| 27 | VIFEGEPMYL |
| 28 | SLFESLEYL |
| 29 | SLLNQPKAV |
| 30 | GLAEFQENV |
| 31 | KLLAVIHEL |
| 32 | TLHDQVHLL |
| 33 | TLYNPERTITV |
| 34 | KLQEKIQEL |
| 35 | SVLEKEIYSI |
| 36 | RVIDDSLVVGV |
| 37 | VLFGELPAL |
| 38 | GLVDIMVHL |
| 39 | FLNAIETAL |
| 40 | ALLQALMEL |
| 41 | ALSSSQAEV |
| 42 | SLITGQDLLSV |
| 43 | QLIEKNWLL |
| 44 | LLDPKTIFL |
| 45 | RLHDENILL |
| 46 | YTFSGDVQL |
| 47 | GLPSATTTV |
| 48 | GLLPSAESIKL |
| 49 | KTASINQNV |
| 50 | SLLQHLIGL |
| 51 | YLMDDFSSL |
| 52 | LMYPYIYHV |
| 53 | KVWSDVTPL |
| 54 | LLWGHPRVALA |
| 55 | VLDGKVAVV |
| 56 | GLLGKVTSV |
| 57 | KMISAIPTL |
| 58 | GLLETTGLLAT |
| 59 | TLNTLDINL |
| 60 | VIIKGLEEI |
| 61 | YLEDGFAYV |
| 62 | KIWEELSVLEV |
| 63 | LLIPFTIFM |
| 64 | ISLDEVAVSL |
| 65 | KISDFGLATV |
| 66 | KLIGNIHGNEV |
| 67 | ILLSVLHQL |
| 68 | LDSEALLTL |
| 69 | VLQENSSDYQSNL |
| 70 | HLLGEGAFAQV |
| 71 | SLVENIHVL |
| 72 | YTFSGDVQL |
| 73 | SLSEKSPEV |
| 74 | AMFPDTIPRV |
| 75 | FLIENLLAA |
| 76 | FTAEFLEKV |
| 77 | ALYGNVQQV |
| 78 | LFQSRIAGV |
| 79 | ILAEEPIYIRV |
| 80 | FLLEREQLL |
| 81 | LLLPLELSLA |
| 82 | SLAETIFIV |
| 83 | AILNVDEKNQV |
| 84 | RLFEEVLGV |
| 85 | YLDEVAFML |
| 86 | KLIDEDEPLFL |
| 87 | KLFEKSTGL |
| 88 | SLLEVNEASSV |
| 89 | GVYDGREHTV |
| 90 | GLYPVTLVGV |
| 91 | ALLSSVAEA |
| 92 | TLLEGISRA |
| 93 | SLIEESEEL |
| 94 | ALYVQAPTV |
| 95 | KLIYKDLVSV |
| 96 | ILQDGQFLV |
| 97 | SLLDYEVSI |
| 98 | LLGDSSFFL |
| 99 | VIFEGEPMYL |
| 100 | ALSYILPYL |
| 101 | FLFVDPELV |
| 102 | SEWGSPHAAVP |
| 103 | ALSELERVL |
| 104 | SLFESLEYL |
| 105 | KVLEYVIKV |
| 106 | VLLNEILEQV |
| 107 | SLLNQPKAV |
| 108 | KMSELQTYV |
| 109 | ALLEQTGDMSL |
| 110 | VIIKGLEEITV |
| 111 | KQFEGTVEI |
| 112 | KLQEEIPVL |
| 113 | GLAEFQENV |
| 114 | NVAEIVIHI |
| 115 | ALAGIVTNV |
| 116 | NLLIDDKGTIKL |
| 117 | VLMQDSRLYL |
| 118 | KVLEHVVRV |
| 119 | LLWGNLPEI |
| 120 | SLMEKNQSL |
| 121 | KLLAVIHEL |
| 122 | ALGDKFLLRV |
| 123 | FLMKNSDLYGA |
| 124 | KLIDHQGLYL |
| 125 | GPGIFPPPPPQP |
| 126 | ALNESLVEC |
| 127 | GLAALAVHL |
| 128 | LLLEAVWHL |
| 129 | SIIEYLPTL |
| 130 | TLHDQVHLL |
| 131 | SLLMWITQC |
| 132 | FLLDKPQDLSI |
| 133 | YLLDMPLWYL |
| 134 | GLLDCPIFL |
| 135 | VLIEYNFSI |
| 136 | TLYNPERTITV |
| 137 | AVPPPPSSV |
| 138 | KLQEELNKV |
| 139 | KLMDPGSLPPL |
| 140 | ALIVSLPYL |
| 141 | FLLDGSANV |
| 142 | ALDPSGNQLI |
| 143 | ILIKHLVKV |
| 144 | VLLDTILQL |
| 145 | HLIAEIHTA |
| 146 | SMNGGVFAV |
| 147 | MLAEKLLQA |
| 148 | YMLDIFHEV |
| 149 | ALWLPTDSATV |
| 150 | GLASRILDA |
| 151 | ALSVLRLAL |
| 152 | SYVKVLHHL |
| 153 | VYLPKIPSW |
| 154 | NYEDHFPLL |
| 155 | VYIAELEKI |
| 156 | VHFEDTGKTLLF |
| 157 | VLSPFILTL |
| 158 | HLLEGSVGV |
| 159 | ALREEEEGV |
| 160 | KEADPTGHSY |
| 161 | TLDEKVAEL |
| 162 | KIQEILTQV |

Although the invention has been described in some detail by way of illustration and example for purposes of clarity of understanding, it should be understood that certain changes and modifications may be practiced within the scope of the appended claims. Modifications of the above-described modes for carrying out the invention that would be understood in view of the foregoing disclosure or made apparent with routine practice or implementation of the invention to persons of skill in oncology, physiology, immunology, and/or related fields are intended to be within the scope of the following claims.

All publications (e.g., Non-Patent Literature), patents, patent application publications, and patent applications mentioned in this specification are indicative of the level of skill of those skilled in the art to which this invention pertains. All such publications (e.g., Non-Patent Literature), patents, patent application publications, and patent applications are herein incorporated by reference to the same extent as if each individual publication, patent, patent application publication, or patent application was specifically and individually indicated to be incorporated by reference.

### EXAMPLES

### Example 1

### Correlation Between Monocytes and Low T-Cell Yield

The inventors used simulated high monocyte containing healthy-donor PBMC populations to demonstrate the inhibitory effect of monocytes on T-cell activation. A control PBMC population with a natural frequency (about 20%) of monocytes (CD14+ cells) was compared to simulated samples created by adding increasing amounts of monocytes (CD14+ cells) back to CD14-depleted PBMC populations. For example, 10% monocytes, 30% monocytes, 60% monocytes, and 80% monocyte populations were created and tested for CD69+ (%), CD25(%), and hLDL-R+ (%) cells, gated on CD3+CD4+CD8a− cells. The higher the percentage of monocytes present in a cell population, the lower the T-cell yield. No significant correlation or trend was observed with the percentage (amount) of B cells, Natural Killer cells (NK), or Natural Killer T-cells (NKT) present in the starting PMBC population.

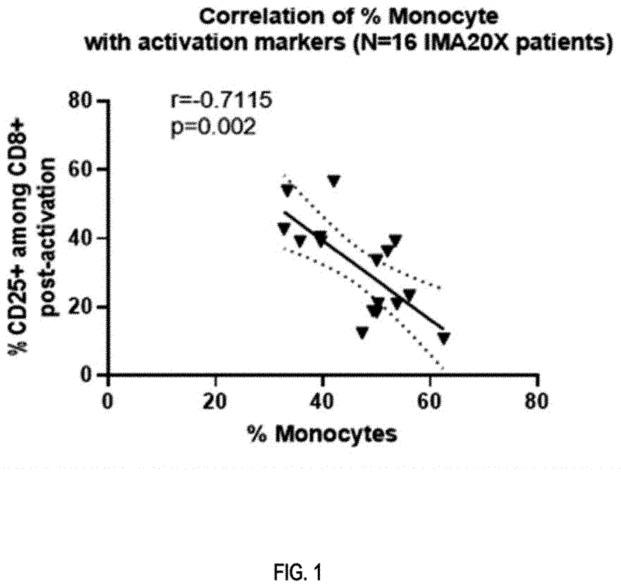

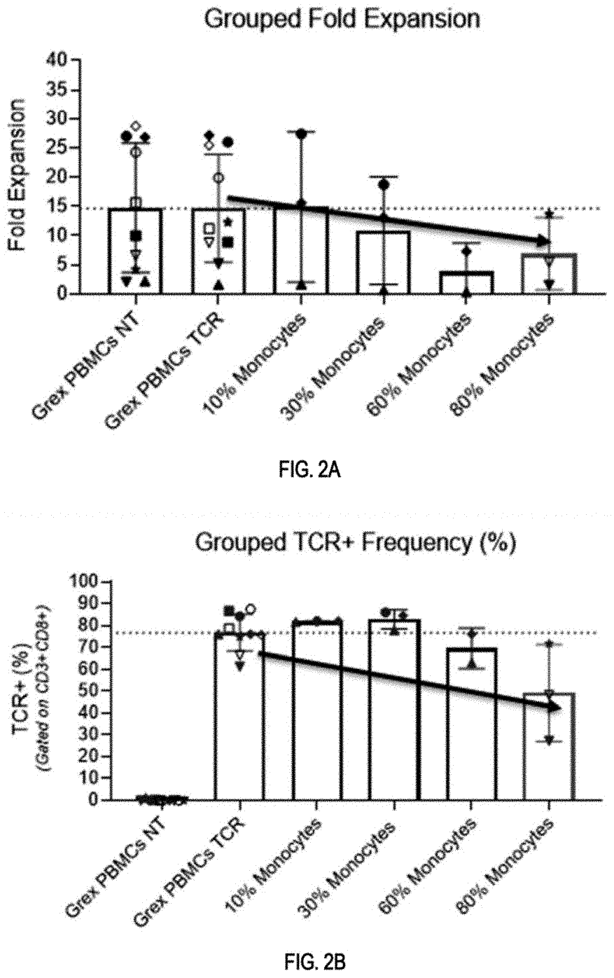

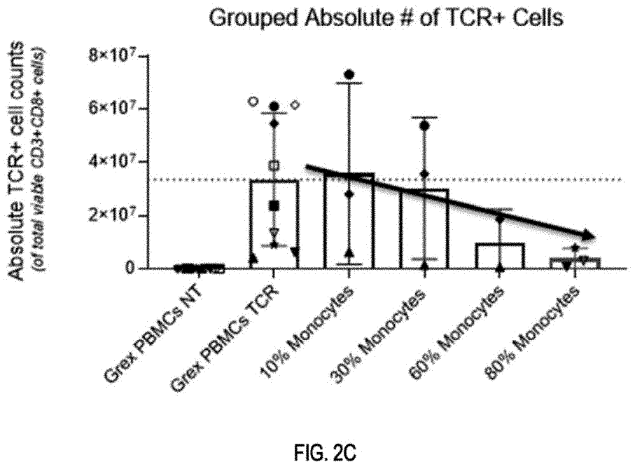

For example, a starting population comprising over 30% monocytes correlated with an over 50% reduction in the T cell yield. See, e.g., FIG. 1. The inventors found that an increased monocyte content 30%) in simulated started PMBC populations negatively impacted manufacturing metrics at Day 6 harvest. FIG. 2A-2C depicts the summary data for 3 mid-scale studies, manufacturing metrics with N=2-10, depending on the condition (N=3-4 healthy donors depending on condition and N-6 for patient samples, depending on condition). The expansion was based on the total number of cells. The poor manufacturing metrics were primarily due to the inhibitory impact on activation and less T cells present at time of transduction.

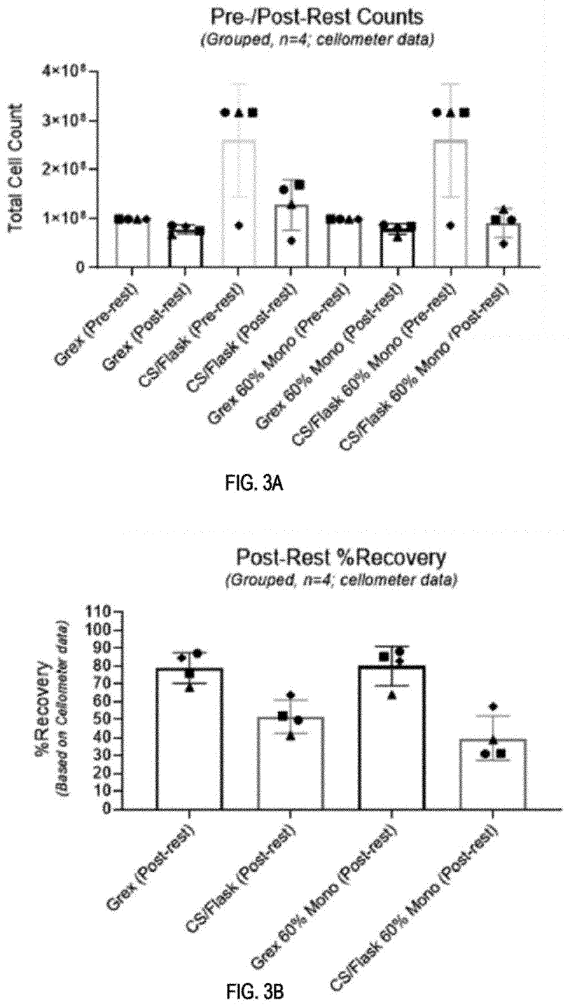

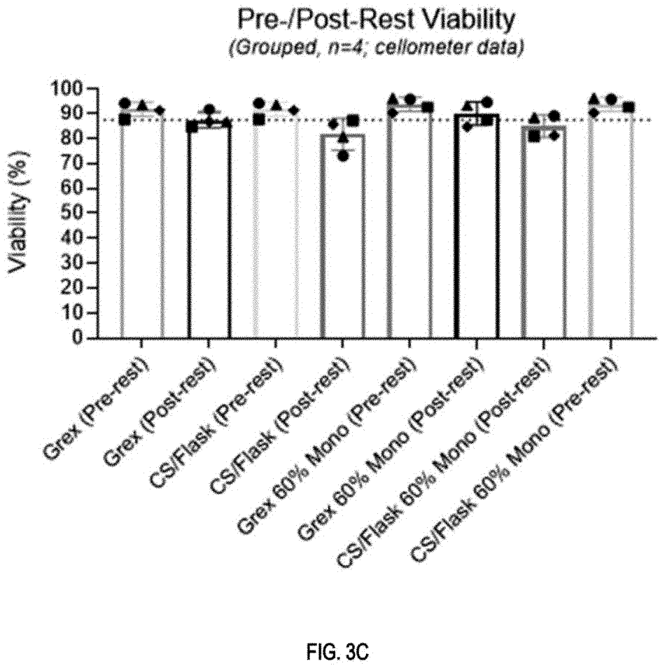

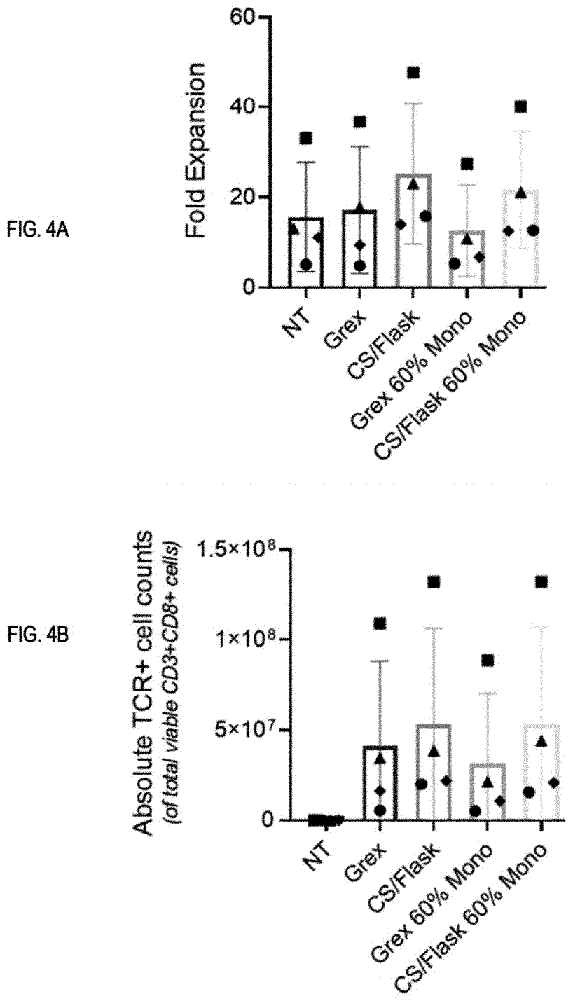

The inventors found that efficient depletion of adherent cells, optionally monocytes, by plastic adherence was indicated by lower post-rest recoveries. FIG. 3A-3C depicts the total cell count (3A), percent recovery (3B), and percent viability (3C) of cells, pre-rest and post-rest with different percentages of monocytes including 60% monocytes. There was an increased fold expansion and higher TCR+ cell yield with plastic adherence-mediated depletion of monocytes. FIG. 4A-B. These results suggest that the presence of a higher amount of monocytes, e.g., 30% or greater, has a deleterious effect on the yield of TCR-transformed T-cells that may be used in immunotherapy. To account for this, the inventors found that reducing the number of adherent cells, including monocytes, leads to an improved yield of TCR-transformed T-cells in each batch.

### Example 2

### Evaluation of Monocyte Depletion Using Plastic Adherence in Cancer Patients

To evaluate the effect of monocyte depletion using plastic adherence as described herein in cancer patients, a total of 13 patients were tested. Four patients using optimized rest and expansion conditions and nine patients using non-optimized conditions.

TABLE 3
Rest Conditions

| | Optimized Conditions | Non-Optimized Conditions |
|---|---|---|
| Rest (plastic | 0.5-0.8 × 10⁶ cells/cm² | No standard seeding density |
| adherence) | for 2 hours | for 2-4 hours |
| Expansion | Grex6M (tall) | Grex6 |
| | Surface Area - 10 cm² | Surface Area - 10 cm² |
| | Media Height - 100 mL | Media Height - 40 mL |
| | Cytokine - 2.5X (total | Cytokine - 1X (total amount) |
| | amount) | |
| Day of | Day 7 | Day 6-10 |
| Harvest | | (variable depending on the |
| | | process) |

PBMC Isolation, Monocyte Depletion and Manufacturing of Engineered T Cell Product

The study design for the evaluation of monocyte depletion using plastic adherence in patients comprised coating bags (Day −1), Thaw, Rest, & Activate (Day 0) [Conditions at rest (A) Grex control and (B) Optimized monocyte depletion by plastic adherence (CellStack)], Transduce in Grex6M (Day 1), Feed (Day 2), Harvest (Day 6-10, preferably Day 7). The cell growth area may vary depending on the size cellstack. For example, 1-layer cellstack may have about 636 cm², 2-layer cellstack may have 1,272 cm² (2×636 cm²), and so on. Grex-10 may have 10 cm². Surface coating of cellstacks were tissue culture treated and Grex were not. Grex may have a thin silicone-based gas permeable membrane at the bottom.

Leukapheresis products from healthy blood donors were obtained from HemaCare. Manufacturing of engineered T cell product from PBMC was performed at different scales in multiple studies. A brief description of each step performed is described below:

PBMC Isolation

PBMC were isolated from the leukapheresis unit via ficoll using closed and automated Sepax C-pro system and NeatCell C-Pro Software (GE Healthcare Life Sciences) according to the manufacturer's recommendations.

Monocyte Depletion and Rest

Monocyte depletion was performed by plastic adherence using either flasks or Cellstacks at 0.5-0.8×10e6 cells/cm2 seeding density. Cells were rested in these vessels for 2-4 hours at 37 C after which non-adherent cells were collected by gently rocking the vessels a few times and then decanting or pipetting out the cells in solution. As a control, some PBMCs were rested in Grex10 for 4 hours at 37 C. These rested cells were then activated. To check for residual cells in the cellstacks, adherent cells were detached using cold PBS containing 10% human AB serum, stained using antibodies and analysed by flow cytometry.

Activation

The 750-AC or 290-AC bags (Saint-Gobain) were coated with anti-human CD3 (0.5 μg/ml) and anti-human CD28 (0.5 μg/ml) antibodies for 16-24 h at 4° C. Freshly prepared PBMC were placed in the anti-CD3/28 coated bags at the concentration of 2×10⁶/ml in complete TexMACS media (supplemented with 5% human AB serum) without cytokines at 37° C. for 16-20 h.

Transductions

Anti-CD3/CD28-activated PBMC were harvested and counted after 16-24 h. Activated PBMC were mixed with the transduction cocktail containing; lentivirus encoding IMA203 or IMA202 TCR (2.5 μl/10⁶ cells) Protamine sulfate (1 μg/ml), IL-7 (10 ng/ml) and IL-15 (50 ng/ml) in serum-free TexMACS media (2×10⁶ cells/ml) in Grex6M or Grex100M for 24 h at 37° C. After 24 h, transduced cells were fed with the TexMACS media containing serum and IL-7 and IL-15 to obtain a final seeding density of 0.5-0.8×10⁶/cm².

Harvest and Cryopreservation

At day 7, transduced and non-transduced cells were harvested, counted and cryopreserved in the CryoStor CS5 Freeze Media. Functional analysis was performed post thawing of the cryopreserved IMA20× products.

The initial data using even the non-optimized conditions showed a benefit in some patients.

Efficient Monocyte Depletion from Patient PBMC by Plastic Adherence using Cellstacks

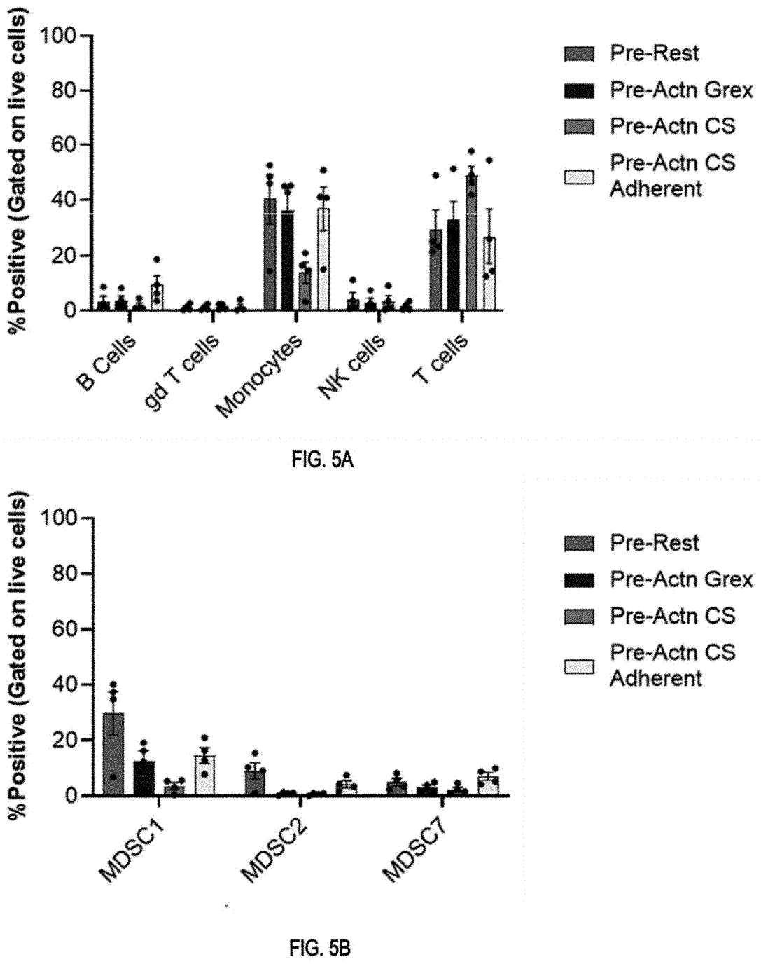

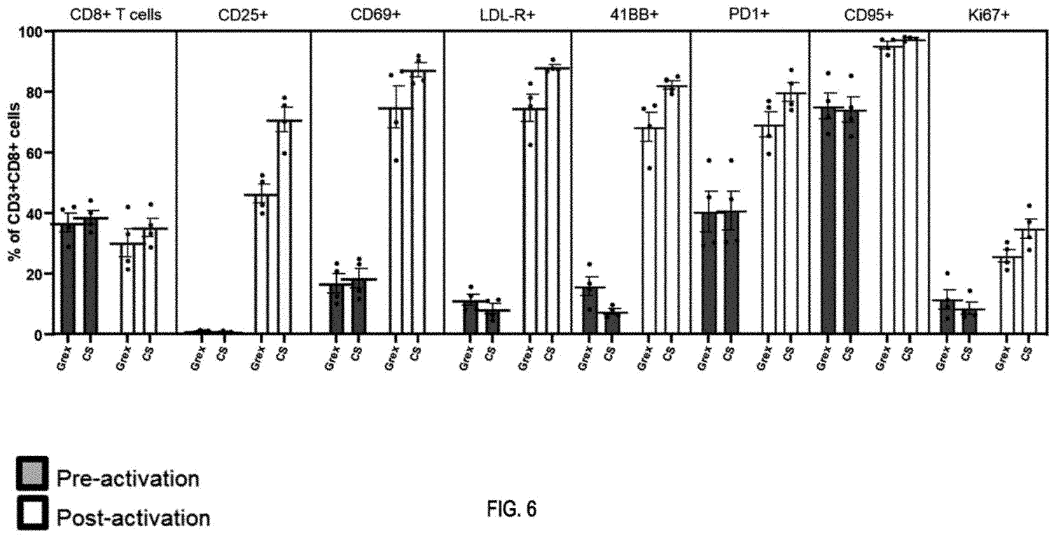

FIGS. 5A and 5B showed efficient monocyte depletion from patient PBMC by plastic adherence using Cellstacks (stacked cell dishes). The depletion of monocytes and myeloid derived suppressor cells (MDSCs) can be achieved efficiently using cellstacks in cancer patient samples. Further, as shown in FIG. 6, increased activation of CD8+ T cells with cell stacks for CD8+ T cells, CD25+ cells, CD69+ cells, LDL-R+ cells, 41BB+ cells, PD1+ cells, CD95+ cells, and Ki67+ cells. A decreased monocyte percentage lead to an increased yield of TCR+CD8+ T cells. For example, a drop of the monocyte percentage below 30% lead to an almost doubling of the CD3+ T-cells.

Increased Fold Expansion and Frequency of CD8+ T cells in Products Generated with Monocyte Depletion by Plastic Adherence

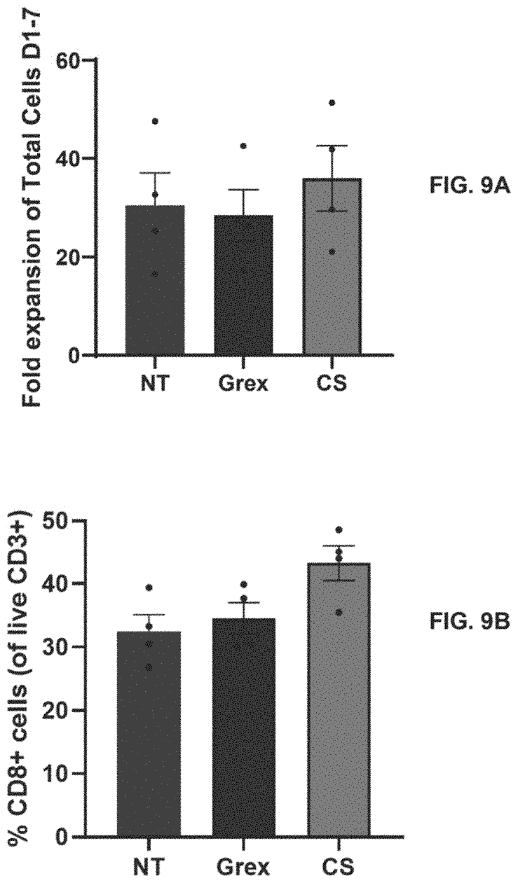

FIG. 9A shows higher fold expansion from transduction to harvest in T cell products prepared by monocyte depletion using plastic adherence (CS) than that using G-Rex. FIG. 9B shows higher % CD8+ cells in T cell products prepared by monocyte depletion using plastic adherence (CS) than that using G-Rex. Non-transduced T cells (NT) serve as controls.

Increased Yield of TCR+CD8+ T cells with Monocyte Depletion

Table 4 shows PBMC populations of the four patients (A-D) using optimized rest and expansion conditions.

TABLE 4

| | PBMC population | | | |
|---|---|---|---|---|
| | A | B | C | D |
| CD3+ (%) | 23.2% | 25.2% | 49% | 21.6% |
| Monocytes (%) | 46% | 48.5% | 14.6% | 52.7% |

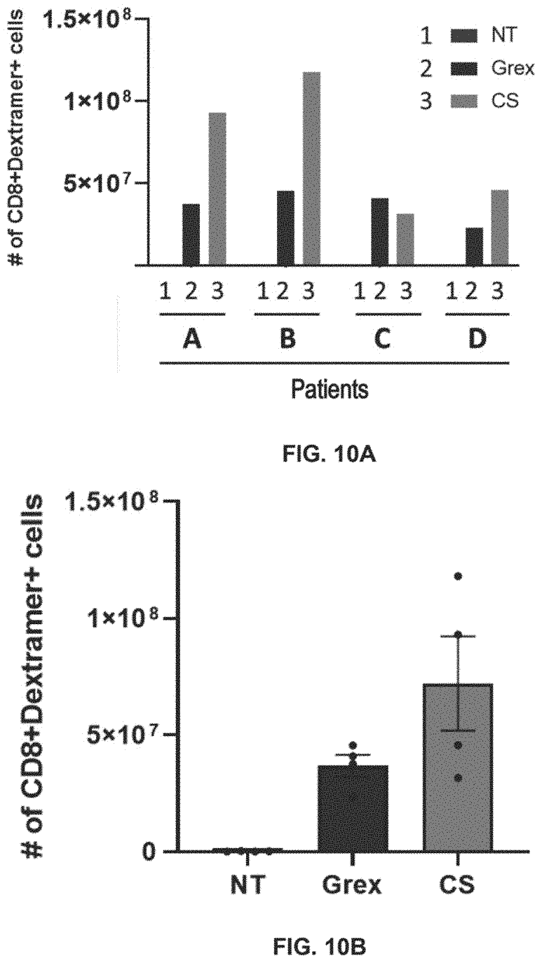

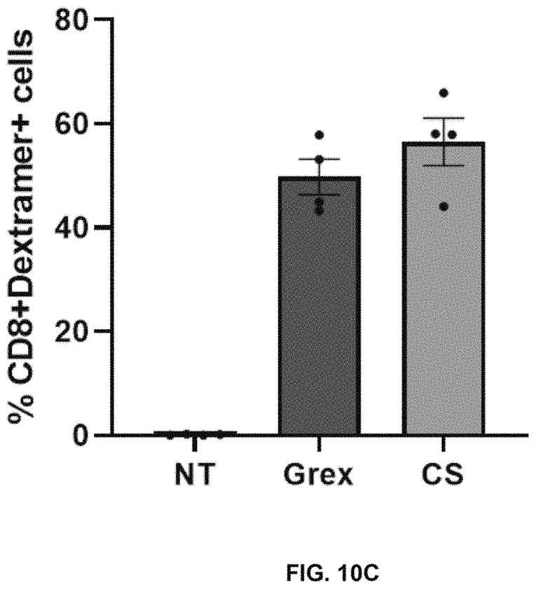

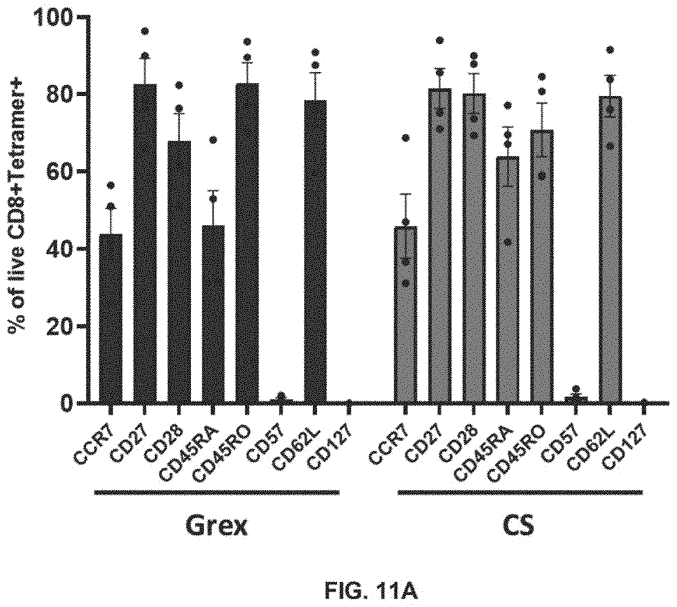

FIG. 10A shows the numbers of TCR+CD8+ T cells prepared by monocyte depletion using plastic adherence (CS) are higher than that using G-Rex in patents A, B, and D, whose % monocytes are high as compared with % CD3+ cells (Table 4). In contrast, monocyte depletion did not significantly increase the numbers of TCR+CD8+ T cells in patent C, whose % monocytes are low as compared with % CD3+ cells (Table 4), suggesting monocyte depletion may be more beneficial by using PBMCs that have high % monocytes in producing high numbers of engineered TCR+CD8+ T cells. FIG. 10B shows the average number of TCR+CD8+ T cells obtained from patents A-D prepared by CS are higher than that using G-Rex. FIG. 10C shows the average % TCR+CD8+ T cells obtained from patents A-D prepared by CS are higher than that using G-Rex. Non-transduced T cells (NT) serve as controls.

Higher Frequency of Naïve T cells in Products Generated with Monocyte Depletion

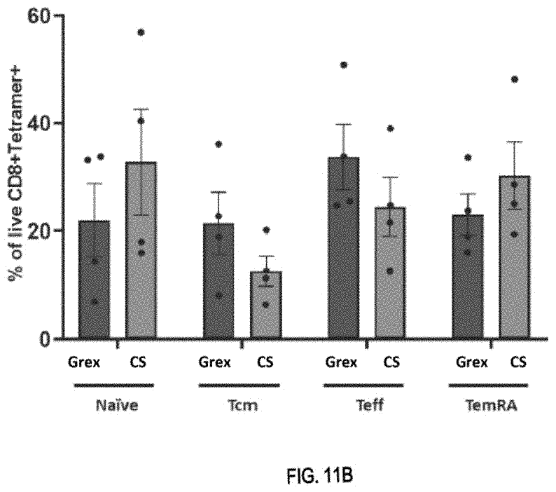

FIG. 11A shows that TCR+CD8+ T cells prepared by monocyte depletion using plastic adherence (CS) contained higher % of cells that are CD45RA+ and CD28+ and lower % of cells that are CD45RO+ than that prepared by using G-Rex, suggesting that more naïve T cells were generated by using CS than by using G-rex. Consistently, FIG. 11B shows that TCR+CD8+ T cells prepared by using CS contained higher % of naïve T cells than that prepared by using G-Rex.

Monocyte Depletion does not Impact Functionality of T Cell Products

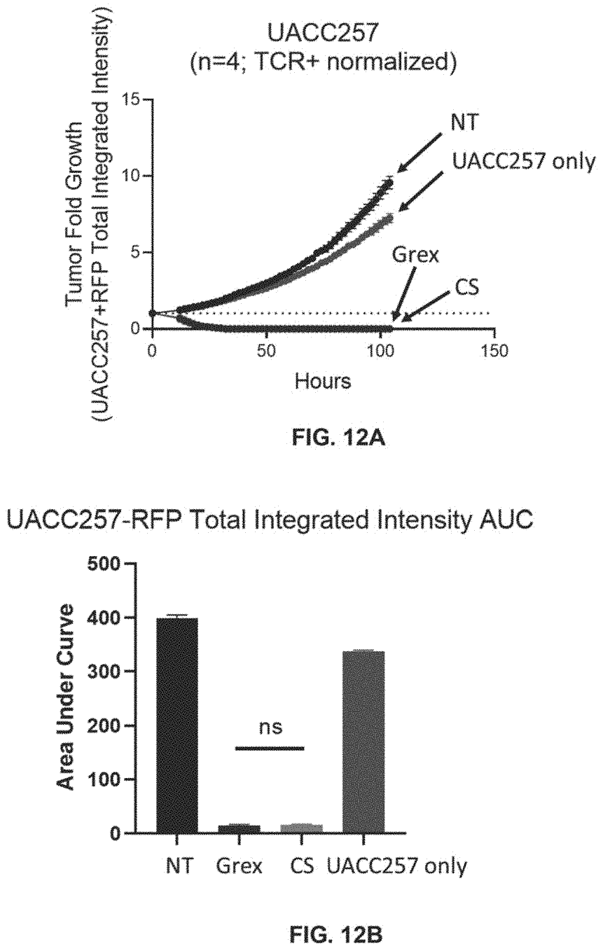

To determine the effect of monocyte depletion on the functionality of T cell products, cell killing activity of T cell products generated by CS and G-rex were compared. TCR+CD8+ T cells obtained from the patients listed in Table 4 (n=4) prepared by monocyte depletion using plastic adherence (CS) and Grex were subject to IncuCyte Killing Assay at E:T=3:1. The target cells (T), e.g., UACC257 (human skin melanoma) present the target peptide/MHC molecule complexes on the cell surface. The transduced TCR in the T cells can bind the target peptide/MHC molecule complexes and kill UACC257 cells. FIG. 12A shows that the cell killing activity of TCR+CD8+ T cells prepared by using CS is comparable to that prepared by using Grex. The cell killing activity was further quantified by measuring the area under curve (AUC) of FIG. 12A. FIG. 12B shows no significant difference in cell killing activity between TCR+CD8+ T cells prepared by using CS and Grex These results suggest that monocyte depletion may not impact functionality of T cell products. (ns=not significant, using one-way ANOVA with Sidak's multiple comparisons test).

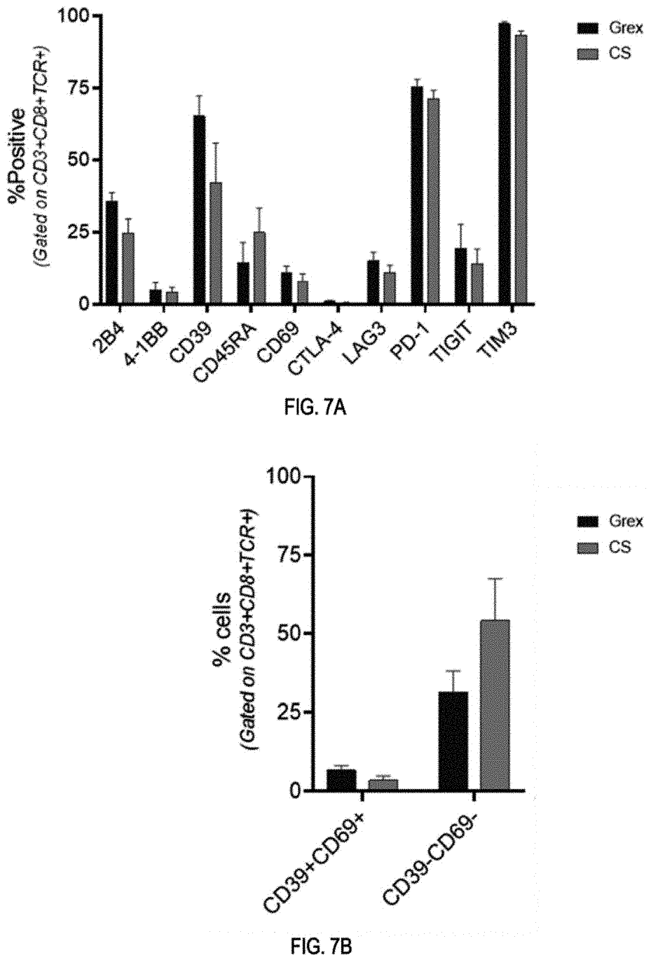

Further, the inventors found a higher frequency of naïve T cells in products generated from monocyte depleted PMBC populations, e.g., CD45RA+, CD28+, and reduced CD45RO+ cells. Additionally, the immune checkpoint inhibitor marker expression was reduced in products generated from monocyte depleted PMBC populations. FIG. 7. This lead to few exhausted cells and a better T-cell product. Further, the monocyte depletion method does not affect the functionality of the T-cell immunotherapy product.

In short, Cellstack (CS)-rest conditions may improve fold expansion and transduction efficiency and significantly improve yield of TCR+CD8+ T cells as compared to Grex-rest conditions. In addition, Cellstack (CS)-rested cells may have significantly more naïve and fewer exhausted cells; while no negative effect on tumor-killing ability was observed.

Combined Patient Data Shows an Improvement in Yield of TCR+CD8+ T Cells at Harvest After Monocyte Depletion

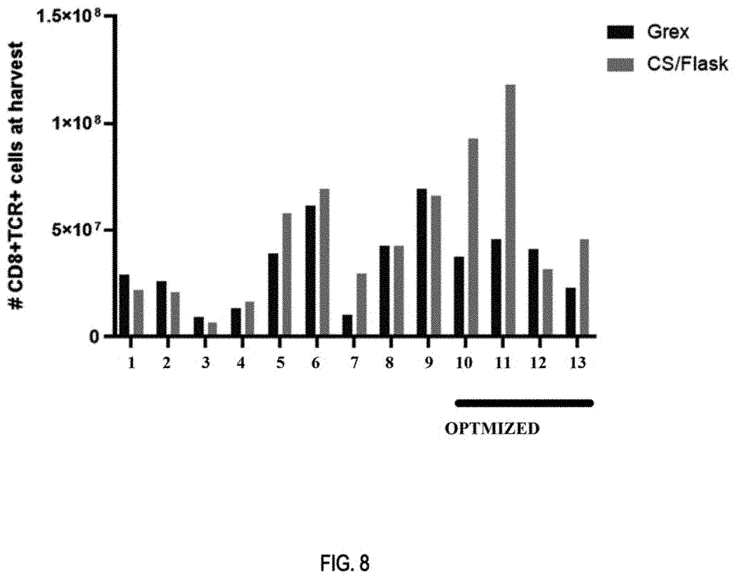

All of the patient data (n=13 and n=4 optimized conditions) showed an improved yield in the TCR+CD8+ T cells at harvest. FIG. 8. A more detailed analysis of the data showed that, out of 13 patients, 7 patients showed increased yields (54%), 2 patients showed no change (15%), and 4 showed reduced yields (31%). When controlling for optimized versus non-optimized, 3 out of 4 of the optimized trials showed an increased yield (75%) and only 4 out of 9 for the non-optimized trials showed an increased yield (45%). This data suggests that the combination of the optimized rest and expansion conditions show the most increase in T-cell yield. The inventors found that the monocyte depletion methods described herein could be successfully scaled-up without negatively impacting the T-cell yield or quality of the T-cell product.

Patients with High Monocyte Frequency are Most Benefitted by Monocyte Depletion Using Optimized Rest and Expansion Conditions

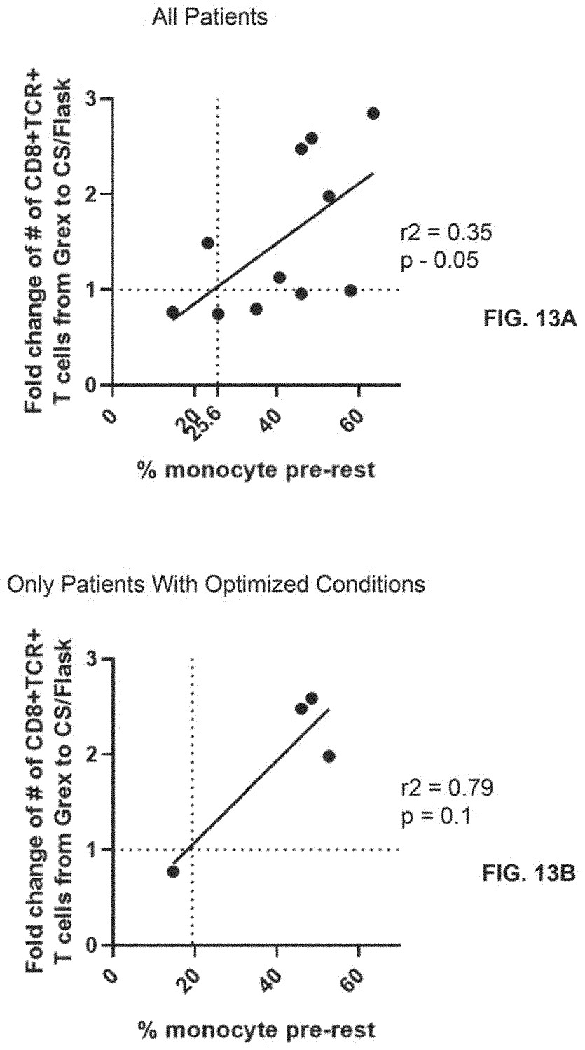

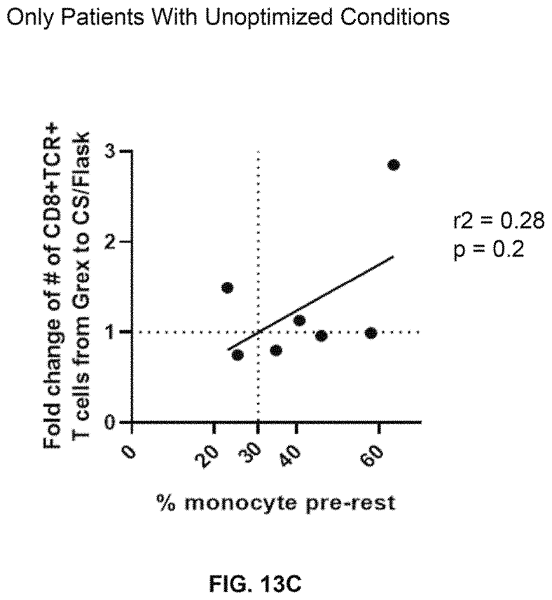

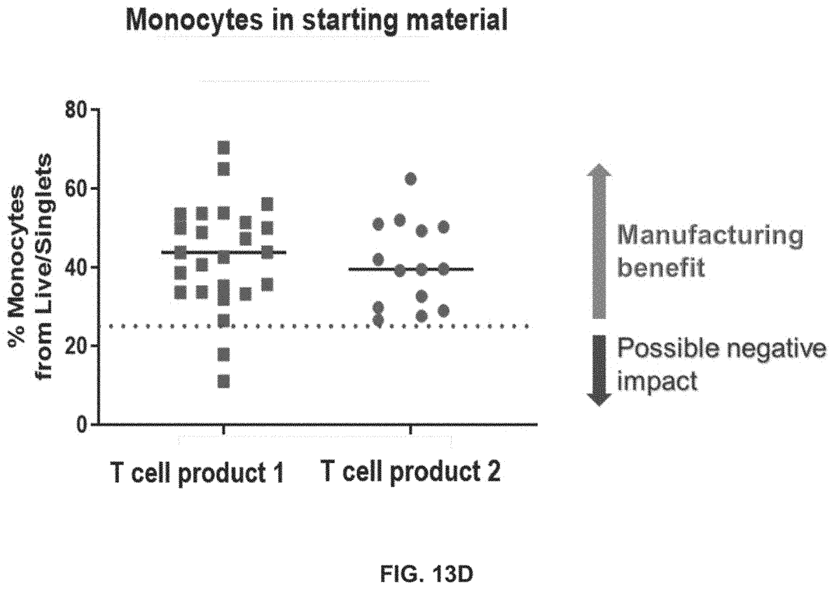

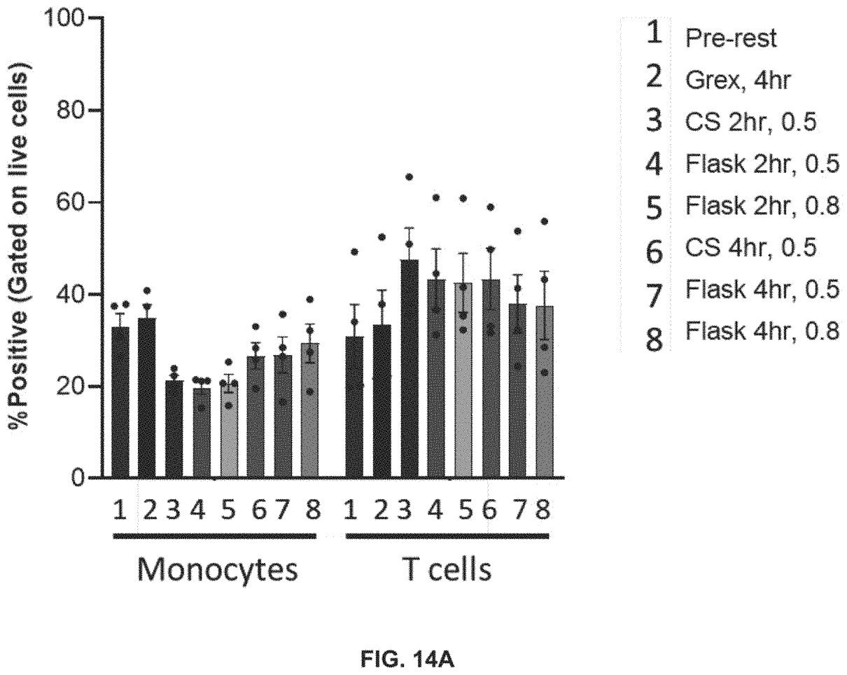

To determine the effect of % monocyte present in patent PBMC on T cell products prepared by using CS and Grex, % monocyte at pre-resting was measured against the fold change of TCR+CD8+ T cells obtained from using Grex to that obtained from using CS. FIG. 13A shows that higher % monocyte present in all patients at pre-rest correlates with higher fold change of CS over Grex (r2=0.35). FIG. 13B shows that higher % monocyte present in only patients with optimized conditions at pre-rest correlates with higher fold change of CS over Grex (r2=0.79). FIG. 13C shows that higher % monocyte present in only patients with unoptimized conditions were measured at pre-rest correlates with higher fold change of CS over Grex (r2=0.28). These results suggest that high fold change in TCR+CD8+ T cells over Grex may be better predicted in only patients with high monocyte frequency with optimized conditions (FIG. 13B). Overall, depleting adherent cell populations during rest led to an improvement in yield of TCR+CD8+ T cells at harvest (1.44-fold increase, n=13). Using optimized rest and expansion conditions led to a more significant improvement in yield of TCR+CD8+ T cells (1.96-fold increase, n=4). FIG. 13D shows that patients with high monocyte frequency (>25% monocytes) in starting materials appear most benefitted by depleting monocytes using optimized rest & expansion conditions.

Efficient and Scalable Depletion of Monocytes Using Optimized Plastic Adherence Method

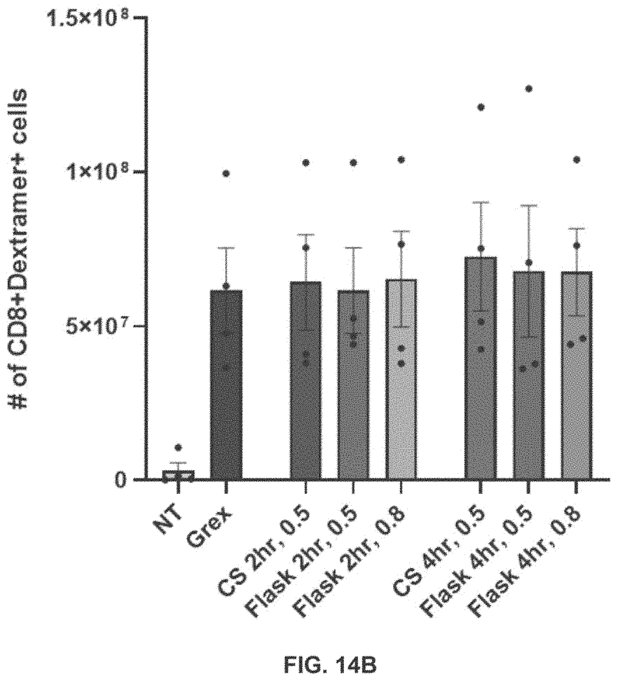

Adherent population depletion can be scaled up from flasks to cellstacks for GMP. For example, FIG. 14A shows that 2 hr rest (3, 4, 5) led to more efficient monocyte depletion than 4 hr (2, 6, 7, 8). Both 0.5 and 0.8×10⁶/cm² seeding densities led to comparable depletion efficiency (4 vs. 5 and 7 vs. 8). In addition, monocyte depletion efficiency in Cellstack (CS)-rested cells appear comparable to that in flasks. Resting for 2 hr by CS appear to have better monocyte depletion efficiency (3 at monocytes) and obtaining higher T cell production (3 at T cells) as compared with that obtaining from resting for 4 hr by CS (6 at monocytes and 6 at T cells). FIG. 14B shows that yield of TCR+CD8+ cells in Cellstack (CS)-rested cells appear comparable to flasks. The yield of TCR+CD8+ cells appear comparable between seeding densities of 0.5 and 0.8×10⁶/cm² using Grex, CS, or flasks.

Corning® CellSTACK® Performed Better at Reducing the Frequency of Monocytes Post-Rest Than Those Obtained from Other Manufactures.

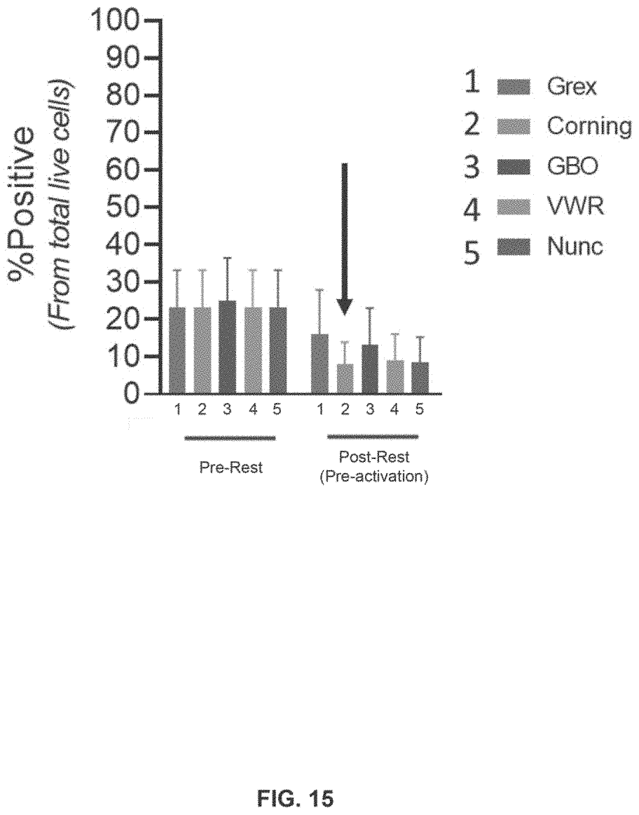

FIG. 15 shows that Corning® CellSTACK® (2) (n=4) performed better in monocyte depletion (as indicated by an arrow) as compared with that obtained from other manufacturers, e.g., from Grex (1) (n=4), GBO (3) (n=3), VWR (4) (n=4), and Nunc (5) (n=4).

Monocyte Depletion is Scalable Using CellStacks

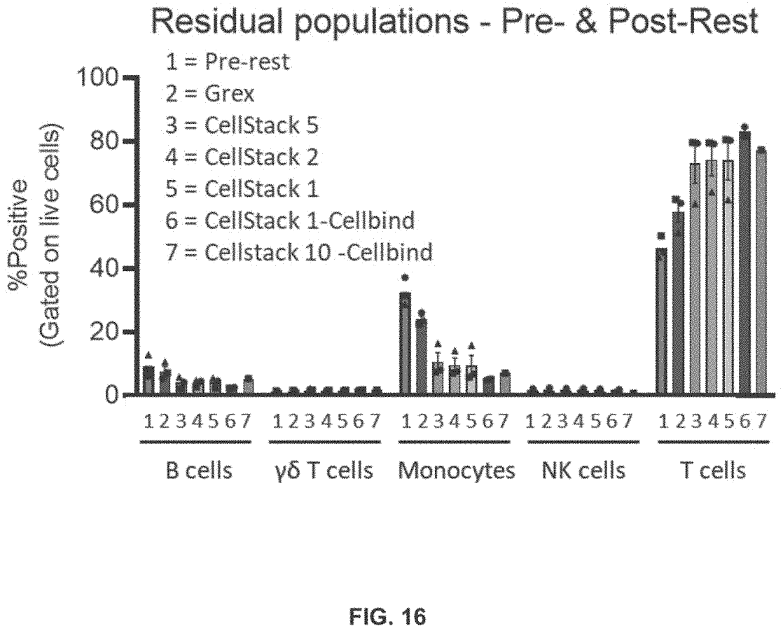

To determine the efficiency of monocyte depletion with increasing cell growth surface, monocyte depletion was performed using Grex, Corning 1-stack (CellStack 1) (with 636 cm² cell growth area), 2-stack (CellStack 2) (with 1,272 cm² cell growth area), 5-stack (CellStack 5) (with 3,180 cm² cell growth area), 1-stack (CellStack 1) with enhanced cell attachment (CellBIND®), and 10-stack (CellStack 10) (with 6,360 cm² cell growth area) with enhanced cell attachment (CellBIND®) were evaluated. FIG. 16 shows that, at post-rest, % monocytes were lower than that at pre-rest, but were comparable among T cells prepared by monocyte depletion using CellStack 1, CellStack 2, CellStack 5, and CellStack 10. At post-rest, % T cells were higher than that at pre-rest, but were comparable among T cells prepared by monocyte depletion using CellStack 1, CellStack 2, CellStack 5, and CellStack 10. These results suggest that increasing cell growth surface, e.g., from 636 cm² cell growth area (CellStack 1) to 6,360 cm² cell growth area (CellStack 10), may not significantly affect the efficiency of monocyte depletion, suggesting the scalability of monocyte depletion using cellstacks.

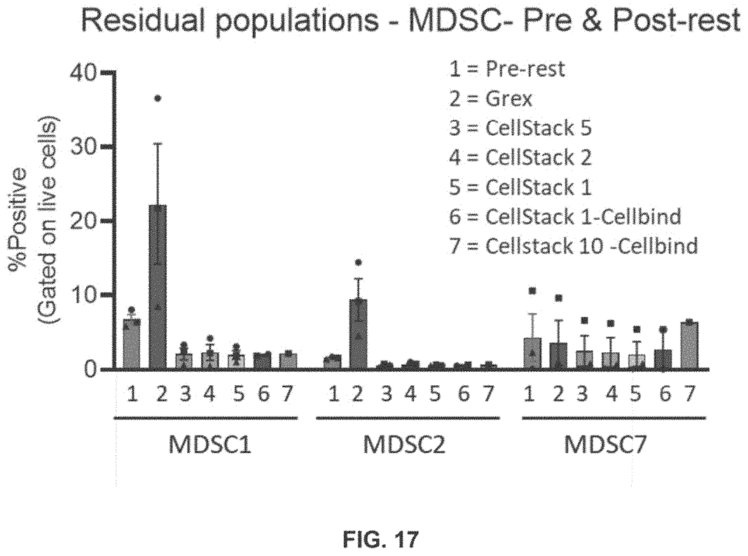

To determine the efficiency of myeloid derived suppressor cells (MDSCs) depletion with increasing cell growth surface, monocyte depletion was performed using Grex, Corning CellStack 1 CellStack 2, CellStack 5 CellStack 1 with enhanced cell attachment (CellBIND®), and CellStack 10 with enhanced cell attachment (CellBIND®) were evaluated. FIG. 17 shows that, at post-rest, % MDSC1 (CD124+CD14+CD3−CD19−CD56−) and MDSC2 (CD124+CD15+CD3−CD19−CD56−) were lower than that at pre-rest. % MDSC7 (CD14−CD15−CD33hiCD3−CD19−CD56−) were reduced from pre-rest to post-rest, but to a less extent as compared with that of MDSC1 and MDSC2. Together, these results suggest that monocyte depletion is scalable using cellstacks.

Effect of Monocyte Depletion on T Cell Products

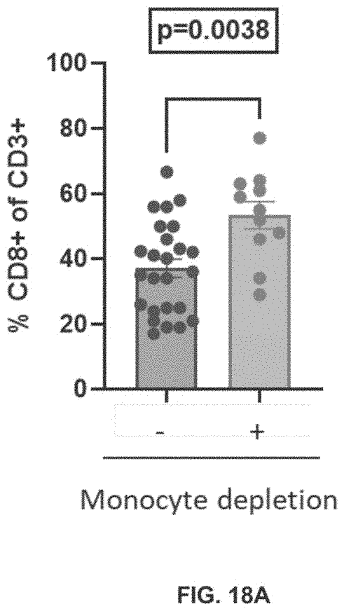

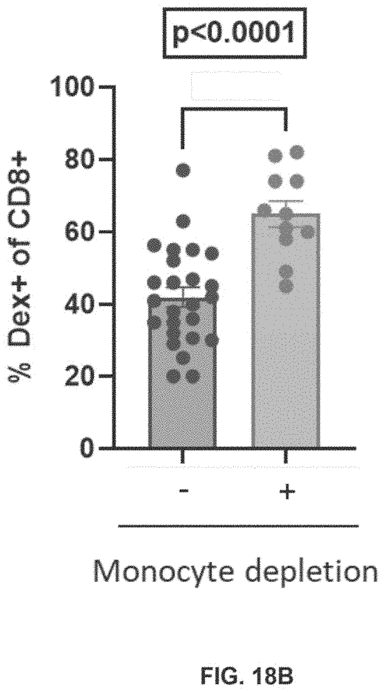

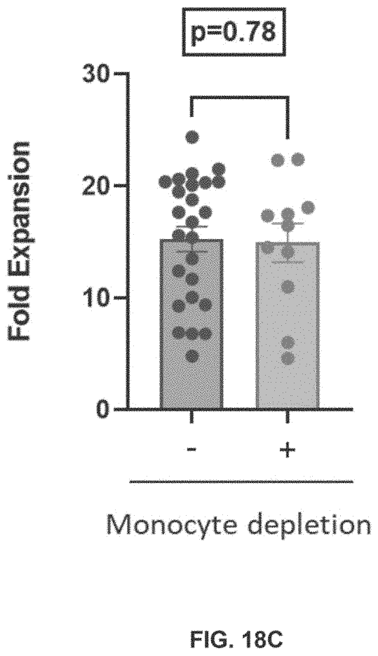

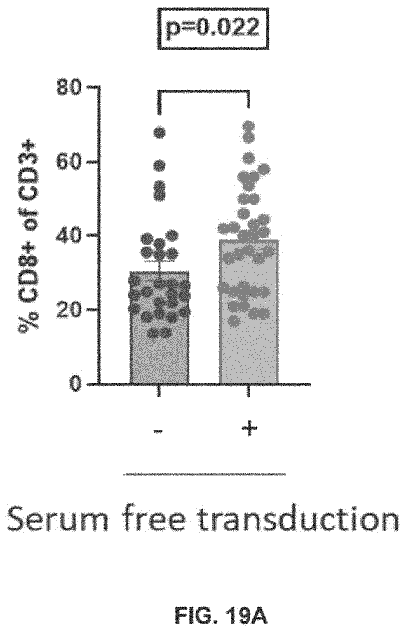

Monocyte depletion in patient T cell manufacturing improves manufacturing metrics. FIG. 18A shows that monocyte depletion increased frequency of CD8 (CD8+CD3+) T cells in T cell products as compared with that without monocyte depletion. FIG. 18B shows that monocyte depletion increased transduction efficiency of exogenous TCR in CD8 (Dex+CD8+) T cells as compared with that without monocyte depletion. FIG. 18C shows that with or without monocyte depletion may have little effect on fold expansion of T cell products.

Effect of Serum Free Transduction on T Cell Products

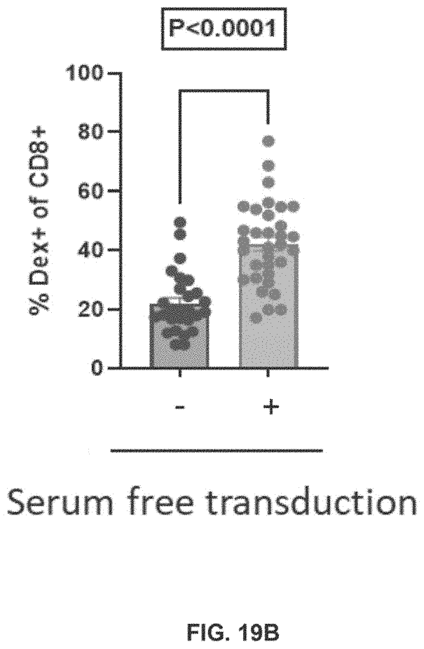

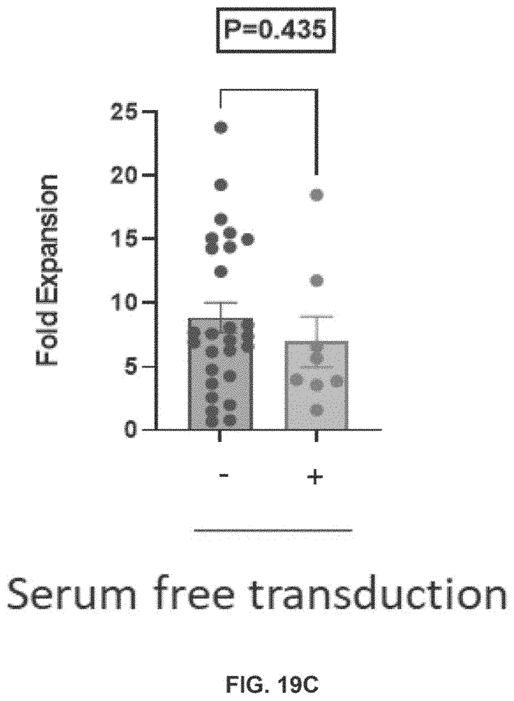

Eliminating serum from transduction step enhances vector integration and transgene expression in GMP scale patient T cell manufacturing runs. FIG. 19A shows that serum free transduction increased frequency of CD8 (CD8+CD3+) T cells in T cell products as compared with transduction in the presence of serum. FIG. 18B shows that serum free transduction increased transduction efficiency of exogenous TCR in CD8 (Dex+CD8+) T cells as compared with transduction in the presence of serum. FIG. 18C shows that with or without serum in transduction may have little effect on fold expansion of T cell products.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the present disclosure is to be limited only by the following claims.

What is claimed is:

1. A method for producing an engineered T cell population, comprising
obtaining a cell population comprising a monocyte and a T cell,
resting the obtained cell population on a surface,
adhering the monocyte to the surface,
retaining a non-adherent cell population,
activating the non-adherent cell population,
introducing a nucleic acid into the activated non-adherent cell population to obtain a transformed T cell, and
expanding the transformed T cell to obtain the engineered T cell population.

2. The method of claim 1, wherein the cell population comprises peripheral blood mononuclear cells (PMBC).

3. The method of claim 1, wherein the monocyte comprises a CD14+ cell.

4. The method of claim 1, wherein the T cell comprises a αβ T cell and/or a γδ T cell.

5. The method of claim 1, wherein the T cell comprises a CD8+ T cell and/or a CD4+ T cell.

6. The method of claim 1, wherein the resting is performed for 2-8 hours.

7. The method of claim 1, wherein the resting is performed at a seeding density of 0.1×10⁶/cm²-2×10⁶/cm².

8. The method of claim 1, wherein the surface comprises a plastic or a glass.

9. The method of claim 8, wherein the plastic comprises polystyrene or polycarbonate.

10. The method of claim 1, wherein the surface comprises a plurality of cell growth areas.

11. The method of claim 10, wherein the plurality of cell growth areas is configured in the form of a plurality of stacks.

12. The method of claim 11, wherein the plurality of stacks comprises at least 2, at least 3, at least 4, at least 5, at least 6, at least 7, at least 8, at least 9, at least 10, at least 15, at least 20, at least 25, at least 30, at least 35, at least 40, at least 45, at least 50 stacks, at least 60 stacks, at least 70 stacks, at least 80 stacks, at least 90 stacks, or at least 100 stacks.

13. The method of claim 10, wherein the plurality of cell growth areas comprises at least 400 cm², at least 500 cm², at least 600 cm², at least 700 cm², at least 800 cm², at least 900 cm², at least 1,000 cm², at least 2,000 cm², at least 3,000 cm², at least 4,000 cm², at least 5,000 cm², at least 6,000 cm², at least 7,000 cm², at least 8,000 cm², at least 9,000 cm², at least 10,000 cm², at least 20,000 cm², at least 30,000 cm², at least 40,000 cm², or at least 50,000 cm².

14. The method of claim 1, wherein the activating is performed in the presence of an anti-CD3 antibody and an anti-CD28 antibody.

15. The method of claim 1, wherein the nucleic acid encodes a recombinant protein.

16. The method of claim 15, wherein the recombinant protein is a chimeric antigen receptor (CAR), a T cell receptor (TCR), a cytokine, an antibody, or a bi-specific binding molecule.

17. The method of claim 16, wherein the recombinant protein is a TCR.

18. The method of claim 17, wherein the TCR binds a peptide in a complex with an MHC molecule.

19. The method of claim 18, wherein the peptide is one selected from SEQ ID NOS: 1-161.

20. The method of claim 18, wherein the MHC molecule is a class I MHC molecule.

21. The method of claim 1, wherein the cell population comprises at least 25% monocyte.

22. The method of claim 1, wherein the non-adherent cell population is a monocyte-deprived cell population.

23. The method of claim 1, wherein the cell population further comprises a myeloid derived suppressor cell (MDSC).

24. The method of claim 23, wherein the MDSC is a CD124+/CD14+/CD3−/CD19−/CD56− cell, a CD124+/CD15+/CD3−/CD19−/CD56− cell, and/or a CD14−/CD15−/CD33 hiCD3−/CD19−/CD56− cell.

25. The method of claim 23, wherein the MDSC is adhered to the surface.

26. A composition comprising the engineered T cell population produced by the method of claim 1.

27. A method of eliciting an immune response in a patient who has cancer, comprising administering to the patient a composition of claim 26, wherein the cancer is hepatocellular carcinoma, colorectal carcinoma, glioblastoma, gastric cancer, esophageal cancer, non-small cell lung cancer, pancreatic cancer, renal cell carcinoma, benign prostate hyperplasia, prostate cancer, ovarian cancer, melanoma, breast cancer, chronic lymphocytic leukemia, Merkel cell carcinoma, small cell lung cancer, non-Hodgkin lymphoma, acute myeloid leukemia, gallbladder cancer and cholangiocarcinoma, urinary bladder cancer, acute lymphocytic leukemia, or uterine cancer.

28. A method of treating a patient who has cancer, comprising administering to the patient a composition of claim 26, wherein the cancer is hepatocellular carcinoma, colorectal carcinoma, glioblastoma, gastric cancer, esophageal cancer, non-small cell lung cancer, pancreatic cancer, renal cell carcinoma, benign prostate hyperplasia, prostate cancer, ovarian cancer, melanoma, breast cancer, chronic lymphocytic leukemia, Merkel cell carcinoma, small cell lung cancer, non-Hodgkin lymphoma, acute myeloid leukemia, gallbladder cancer and cholangiocarcinoma, urinary bladder cancer, acute lymphocytic leukemia, or uterine cancer.

29. The method of claim 1, wherein the introducing a nucleic acid into the activated non-adherent cell population is performed with or without serum.

30. The method of claim 29, wherein the introducing a nucleic acid into the activated non-adherent cell population is performed without serum.

31. The method of claim 1, wherein the nucleic acid further encodes a CD8αβ heterodimer or a CD8α homodimer.

32. The method of claim 31, wherein the CD8α comprises the amino acid sequence selected from SEQ ID NO: 163-166 and the CD8β comprises the amino acid sequence selected from SEQ ID NO: 167-173.

33. The method of claim 1, wherein the nucleic acid further comprises a woodchuck hepatitis virus posttranscriptional responsive element (WPRE) comprising the nucleotide sequence selected from SEQ ID NO: 174-176.
